SEQUENCE LISTING (9 sequences)

SEQ ID NO: 1 - protein, 461 aa, Homo sapiens
MTPNSMTENGLTAWDKPKHCPDREHDWKLVGMSEACLHRKSHSERRSTLKNEQSSPHLIQTTWTSSIFHLDHDDVNDQSVSSAQTFQTEEKKCKGYIPSYLDKDELCVVCGDKATGYHYRCITCEGCKGFFRRTIQKNLHPSYSCKYEGKCVIDKVTRNQCQECRFKKCIYVGMATDLVLDDSKRLAKRKLIEENREKRRREELQKSIGHKPEPTDEEWELIKTVTEAHVATNAQGSHWKQKRKFLPEDIGQAPIVNAPEGGKVDLEAFSHFTKIITPAITRVVDFAKKLPMFCELPCEDQIILLKGCCMEIMSLRAAVRYDPESETLTLNGEMAVTRGQLKNGGLGVVSDAIFDLGMSLSSFNLDDTEVALLQAVLLMSSDRPGLACVERIEKYQDSFLLAFEHYINYRKHHVTHFWPKLLMKVTDLRMIGACHASRFLHMKVECPTELFPPLFLEVFED

SEQ ID NO: 2 - protein, 1153 aa, Homo sapiens
MALRVLLLTALTLCHGFNLDTENAMTFQENARGFGQSVVQLQGSRVVVGAPQEIVAANQRGSLYQCDYSTGSCEPIRLQVPVEAVNMSLGLSLAATTSPPQLLACGPTVHQTCSENTYVKGLCFLFGSNLRQQPQKFPEALRGCPQEDSDIAFLIDGSGSIIPHDFRRMKEFVSTVMEQLKKSKTLFSLMQYSEEFRIHFTFKEFQNNPNPRSLVKPITQLLGRTHTATGIRKVVRELFNITNGARKNAFKILVVITDGEKFGDPLGYEDVIPEADREGVIRYVIGVGDAFRSEKSRQELNTIASKPPRDHVFQVNNFEALKTIQNQLREKIFAIEGTQTGSSSSFEHEMSQEGFSAAITSNGPLLSTVGSYDWAGGVFLYTSKEKSTFINMTRVDSDMNDAYLGYAAAIILRNRVQSLVLGAPRYQHIGLVAMFRQNTGMWESNANVKGTQIGAYFGASLCSVDVDSNGSTDLVLIGAPHYYEQTRGGQVSVCPLPRGQRARWQCDAVLYGEQGQPWGRFGAALTVLGDVNGDKLTDVAIGAPGEEDNRGAVYLFHGTSGSGISPSHSQRIAGSKLSPRLQYFGQSLSGGQDLTMDGLVDLTVGAQGHVLLLRSQPVLRVKAIMEFNPREVARNVFECNDQVVKGKEAGEVRVCLHVQKSTRDRLREGQIQSVVTYDLALDSGRPHSRAVFNETKNSTRRQTQVLGLTQTCETLKLQLPNCIEDPVSPIVLRLNFSLVGTPLSAFGNLRPVLAEDAQRLFTALFPFEKNCGNDNICQDDLSITFSFMSLDCLVVGGPREFNVTVTVRNDGEDSYRTQVTFFFPLDLSYRKVSTLQNQRSQRSWRLACESASSTEVSGALKSTSCSINHPIFPENSEVTFNITFDVDSKASLGNKLLLKANVTSENNMPRTNKTEFQLELPVKYAVYMVVTSHGVSTKYLNFTASENTSRVMQHQYQVSNLGQRSLPISLVFLVPVRLNQTVIWDRPQVTFSENLSSTCHTKERLPSHSDFLAELRKAPVVNCSIAVCQRIQCDIPFFGIQEEFNATLKGNLSFDWYIKTSHNHLLIVSTAEILFNDSVFTLLPGQGAFVRSQTETKVEPFEVPNPLPLIVGSSVGGLLLLALITAALYKLGFFKRQYKDMMSEGGPPGAEPQ

SEQ ID NO: 3 - protein, 1152 aa, Homo sapiens
MALRVLLLTALTLCHGFNLDTENAMTFQENARGFGQSVVQLQGSRVVVGAPQEIVAANQRGSLYQCDYSTGSCEPIRLQVPVEAVNMSLGLSLAATTSPPQLLACGPTVHQTCSENTYVKGLCFLFGSNLRQQPQKFPEALRGCPQEDSDIAFLIDGSGSIIPHDFRRMKEFVSTVMEQLKKSKTLFSLMQYSEEFRIHFTFKEFQNNPNPRSLVKPITQLLGRTHTATGIRKVVRELFNITNGARKNAFKILVVITDGEKFGDPLGYEDVIPEADREGVIRYVIGVGDAFRSEKSRQELNTIASKPPRDHVFQVNNFEALKTIQNQLREKIFAIEGTQTGSSSSFEHEMSQEGFSAAITSNGPLLSTVGSYDWAGGVFLYTSKEKSTFINMTRVDSDMNDAYLGYAAAIILRNRVQSLVLGAPRYQHIGLVAMFRQNTGMWESNANVKGTQIGAYFGASLCSVDVDSNGSTDLVLIGAPHYYEQTRGGQVSVCPLPRGRARWQCDAVLYGEQGQPWGRFGAALTVLGDVNGDKLTDVAIGAPGEEDNRGAVYLFHGTSGSGISPSHSQRIAGSKLSPRLQYFGQSLSGGQDLTMDGLVDLTVGAQGHVLLLRSQPVLRVKAIMEFNPREVARNVFECNDQVVKGKEAGEVRVCLHVQKSTRDRLREGQIQSVVTYDLALDSGRPHSRAVFNETKNSTRRQTQVLGLTQTCETLKLQLPNCIEDPVSPIVLRLNFSLVGTPLSAFGNLRPVLAEDAQRLFTALFPFEKNCGNDNICQDDLSITFSFMSLDCLVVGGPREFNVTVTVRNDGEDSYRTQVTFFFPLDLSYRKVSTLQNQRSQRSWRLACESASSTEVSGALKSTSCSINHPIFPENSEVTFNITFDVDSKASLGNKLLLKANVTSENNMPRTNKTEFQLELPVKYAVYMVVTSHGVSTKYLNFTASENTSRVMQHQYQVSNLGQRSLPISLVFLVPVRLNQTVIWDRPQVTFSENLSSTCHTKERLPSHSDFLAELRKAPVVNCSIAVCQRIQCDIPFFGIQEEFNATLKGNLSFDWYIKTSHNHLLIVSTAEILFNDSVFTLLPGQGAFVRSQTETKVEPFEVPNPLPLIVGSSVGGLLLLALITAALYKLGFFKRQYKDMMSEGGPPGAEPQ

SEQ ID NO: 4 - DNA, 3551 bp, Homo sapiens
cgcacccagttcacgcgccacagctatgtgtccccgagccgcgcgggcgcccgcgacgctactcctcgccctgggcgcggtgctgtggcctgcggctggcgcctgggagcttacgattttgcacaccaacgacgtgcacagccggctggagcagaccagcgaggactccagcaagtgcgtcaacgccagccgctgcatgggtggcgtggctcggctcttcaccaaggttcagcagatccgccgcgccgaacccaacgtgctgctgctggacgccggcgaccagtaccagggcactatctggttcaccgtgtacaagggcgccgaggtggcgcacttcatgaacgccctgcgctacgatgccatggcactgggaaatcatgaatttgataatggtgtggaaggactgatcgagccactcctcaaagaggccaaatttccaattctgagtgcaaacattaaagcaaaggggccactagcatctcaaatatcaggactttatttgccatataaagttcttcctgttggtgatgaagttgtgggaatcgttggatacacttccaaagaaaccccttttctctcaaatccagggacaaatttagtgtttgaagatgaaatcactgcattacaacctgaagtagataagttaaaaactctaaatgtgaacaaaattattgcactgggacattcgggttttgaaatggataaactcatcgctcagaaagtgaggggtgtggacgtcgtggtgggaggacactccaacacatttctttacacaggcaatccaccttccaaagaggtgcctgctgggaagtacccattcatagtcacttctgatgatgggcggaaggttcctgtagtccaggcctatgcttttggcaaatacctaggctatctgaagatcgagtttgatgaaagaggaaacgtcatctcttcccatggaaatcccattcttctaaacagcagcattcctgaagatccaagcataaaagcagacattaacaaatggaggataaaattggataattattctacccaggaattagggaaaacaattgtctatctggatggctcctctcaatcatgccgctttagagaatgcaacatgggcaacctgatttgtgatgcaatgattaacaacaacctgagacacgcggatgaaacgttctggaaccacgtatccatgtgcattttaaatggaggtggtatccggtcgcccattgatgaacgcaacaatggcacaattacctgggagaacctggctgctgtattgccctttggaggcacatttgacctagtccagttaaaaggttccaccctgaagaaggcctttgagcatagcgtgcaccgctacggccagtccactggagagttcctgcaggtgggcggaatccatgtggtgtatgatctttcccgaaaacctggagacagagtagtcaaattagatgttctttgcaccaagtgtcgagtgcccagttatgaccctctcaaaatggacgaggtatataaggtgatcctcccaaacttcctggccaatggtggagatgggttccagatgataaaagatgaattattaagacatgactctggtgaccaagatatcaacgtggtttctacatatatctccaaaatgaaagtaatttatccagcagttgaaggtcggatcaagttttccacaggaagtcactgccatggaagcttttctttaatatttctttcactttgggcagtgatctttgttttataccaatagccaaaaattctccttgcctttaatgtgtgaaactgcattttttcaagtgagattcaaatctgccttttaggacctggctttgtgacagcaaaaaccatctttacaggctcctagaagctgaaggttagagcattataaaatgaagagacagacatgattactcagggtcagcaacctagtgagttagaaaaaaaattaacatagggccctataaggagaaagccaactatgttaagtttatgtgtccaaattttaatgaaattttactaacaattttaaaccatatttttcttcttcatatccatttctaatccatcaaacagcttatgtttacataaaattttatcattcacaaggaagttttaagcacactgtctcatttgatatccacaacttatttttggtaggaaagagagatgtttttcccacctgtcagatgaaaaaactgaagctcaaaaagggttgacttgaccatacagctaatgctgacagatccaagacctagacctaggtcttttgaactcaagtccaggattctcaactatatcaagttactgttcagaatacttaatatctcctctcttcataattatcaatagccccaagctcatggatgacaaatctctgctttatttcttgtctctattttttcactttatagctcctgttataatagcaagtttaatggtataaacacaggataccatcctctcttgcaacacccatgtgcctttgatgagtcaggtagcaagctgtagtagataatgagaaaggccagaggctgcaaaagacagtcaaaggacacgagagaaaggaaggggaagaacaggactccaggactgttttatattatagaaaagcaagagctaaagagcatttacacatgttaaacagatacttgttaagcatagtgcctgacacacggcattagctgttatgagattccatcagctctgcctctgtcctctttcttctaacatgaaggtatcatgagaagagaaccttctaacataagctgtaattctaaacctgcacttgtccctctccagcaagaggctagcactgaattcattctactcatactacacacccagttatggaatgtccagagttctcgaagaaaataaatgactttaggaagaggtatacattttttaagtcgctctgcctccaaatctgaacagtcactgtaaatcattcttaagcccagatatgagaacttctgctggaaagtgggaccctctgagtgggtggtcagaaaatacccatgctgatgaaatgacctatgcccaaagaacaaatacttaacgtgggagtggaaccacatgagcctgctcagctctgcataagtaattcaagaaatgggaggcttcaccttaaaaacagtgtgcaaatggcagctagaggttttgataggaagtatgtttgtttcttagtgtttacaaatattaagtactcttgatacaaaatatacttttaaacttcataacctttttataaaagttgttgcagcaaaataatagcctcggttctatgcatatatggattagctataaaaaatgtcaataagattgtacaaggaaaattagagaaaggcacatttagggtttattttttacacttggccagtaaaatagggtaaatcctattagaattttttaaagaactttttttaagtttcctaaatctgtgtgtgtattgtgaagtggtataagaaatgactttgaaccactttgcaattgtagattcccaacaataaaattgaagataaaaaaaaaaaaaaaaaaaaaaaaaaaaaaaa

SEQ ID NO: 5 - protein, 574 aa, Homo sapiens [sequence omitted]

SEQ ID NO: 6 - protein, 388 aa, Homo sapiens
MSKVFKKTSSNGKLSIYLGKRDFVDHVDTVEPIDGVVLVDPEYLKCRKLFVMLTCAFRYGRDDLEVIGLTFRKDLYVQTLQVVPAESSSPQGPLTVLQERLLHKLGDNAYPFTLQMVTNLPCSVTLQPGPEDAGKPCGIDFEVKSFCAENPEETVSKRDYVRLVVRKVQFAPPEAGPGPSAQTIRRFLLSAQPLQLQAWMDREVHYHGEPISVNVSINNCTNKVIKKIKISVDQITDVVLYSLDKYTKTVFIQEFTETVAANSSFSQSFAVTPILAASCQKRGLALDGKLKHEDTNLASSTIIRPGMDKELLGILVSYKVRVNLMVSCGGILGDLTASDVGVELPLVLIHPKPSHEAASSEDIVIEEFTRKGEEESQKAVEAEGDEGS

SEQ ID NO: 7 - protein, 364 aa, Homo sapiens
MAQQWSLQRLAGRHPQDSYEDSTQSSIFTYTNSNSTRGPFEGPNYHIAPRWVYHLTSVWMIFVVIASVFTNGLVLAATMKFKKLRHPLNWILVNLAVADLAETVIASTISVVNQVYGYFVLGHPMCVLEGYTVSLCGITGLWSLAIISWERWMVVCKPFGNVRFDAKLAIVGIAFSWIWAAVWTAPPIFGWSRYWPHGLKTSCGPDVFSGSSYPGVQSYMIVLMVTCCITPLSIIVLCYLQVWLAIRAVAKQQKESESTQKAEKEVTRMVVVMVLAFCFCWGPYAFFACFAAANPGYPFHPLMAALPAFFAKSATIYNPVIYVFMNRQFRNCILQLFGKKVDDGSELSSASKTEVSSVSSVSPA

SEQ ID NO: 8 - protein, 364 aa, Homo sapiens
MAQQWSLQRLAGRHPQDSYEDSTQSSIFTYTNSNSTRGPFEGPNYHIAPRWVYHLTSVWMIFVVTASVFTNGLVLAATMKFKKLRHPLNWILVNLAVADLAETVIASTISIVNQVSGYFVLGHPMCVLEGYTVSLCGITGLWSLAIISWERWMVVCKPFGNVRFDAKLAIVGIAFSWIWAAVWTAPPIFGWSRYWPHGLKTSCGPDVFSGSSYPGVQSYMIVLMVTCCIIPLAIIMLCYLQVWLAIRAVAKQQKESESTQKAEKEVTRMVVVMIFAYCVCWGPYTFFACFAAANPGYAFHPLMAALPAYFAKSATIYNPVIYVFMNRQFRNCILQLFGKKVDDGSELSSASKTEVSSVSSVSPA

SEQ ID NO: 9 - protein, 348 aa, Homo sapiens [sequence omitted]

(19) United States
(12) Patent Application Publication
(10) Pub. No.: US 2023/0090462 A1
(43) Pub. Date: Mar. 23, 2023

(54) ISOLATION, ENRICHMENT AND EXPANSION OF CONE PROGENITOR CELLS AND USES THEREOF

(71) Applicants: The Schepens Eye Research Institute, Inc., Boston, MA (US); Massachusetts Eye and Ear Infirmary, Boston, MA (US); Massachusetts Institute of Technology, Cambridge, MA (US)

(72) Inventors: Michael J. Young, Essex, MA (US); Pierre Colombe, Cambridge, MA (US); Deepti Singh, Melrose, MA (US)

(21) Appl. No.: 17/439,251

(22) PCT Filed: Mar. 16, 2020
(86) PCT No.: PCT/US2020/022929

§ 371 Date: Sep. 14, 2021

Related U.S. Application Data
(60) Provisional application No. 62/819,160, filed on Mar. 15, 2019.

(51) Int. Cl. A61K 35/545; C12N 5/0793; A61P 27/02; A61K 9/00; A61K 49/00

(52) U.S. Cl. CPC A61K 35/545; C12N 5/062; A61P 27/02; A61K 9/0019; A61K 9/0048; A61K 49/0008; C12N 2506/02; C12N 2500/44; C12N 2500/32; C12N 2501/115

(57) ABSTRACT

Progenitor cells were isolated, purified and expanded using a microfluidic based cell sorting approach. The methods were successfully in purifying cone progenitor cells (CPCs) defined based on a proliferative population expressing cone arrestin and Red/Green (R/G) opsin at greater than 80% using a two multistage approach.

### CROSS REFERENCE TO RELATED APPLICATIONS

This Application is a national stage application under 35 U.S.C. § 371 of International Patent Application No. PCT/US2020/022929, filed Mar. 16, 2020, which claims the benefit of and priority under 35 U.S.C. § 119(e) to U.S. Provisional Application 62/819,160 filed on Mar. 15, 2019, the entire contents of which are hereby expressly incorporated by reference herein.

### INCORPORATION BY REFERENCE OF SEQUENCE LISTING

The content of the text file named “036770-581001WO_SL.TXT”, which was created on Apr. 29, 2020, and is 46,671 bytes in size, is hereby incorporated by reference in its entirety.

### FIELD OF THE DISCLOSURE

The disclosure relates to cells and methods for treatment of retinal degenerative disease.

### BACKGROUND

Retinal degenerative diseases, which lead to the death of rod and cone photoreceptor cells, are the leading cause of inherited vision loss worldwide. Induced pluripotent or embryonic stem cells (iPSCs/ESCs) have been proposed as a possible source of new photoreceptors to restore vision in these conditions. Studies carried out in mouse models of retinal degeneration over the past decade have highlighted several limitations for cell replacement in the retina, such as the low efficiency of cone photoreceptor production from stem cell cultures and the poor integration of grafted cells in the host retina.

### SUMMARY

The invention provides a solution to the limitations and drawbacks associated with previous approaches in the isolation, purification and enrichment of progenitor cells. For example, the invention provides a purified or enriched population of photoreceptor precursor cells, for example, cone precursor photoreceptor cells (CPCs), methods of producing these cells and use of the cells for the treatment of ocular disorders, e.g., retinal degenerative diseases, and other diseases. The invention encompasses methods for the isolation, purification and expansion of progenitor cells using a microfluidic based cell sorting approach. The method is a scalable GMP capable protocol for production, e.g. of human cones, providing a unique ability to create universal, allogenic, cone photoreceptor cells for preserving and restoring vision.

Accordingly, in certain embodiments, a composition comprising a purified population of cells, wherein the cells are CD73⁺, Thyroid Hormone Receptor beta (Thrb⁺), CD 11b⁻. In this and other embodiments, the purified population of cells are derived from: embryonic retinas, embryonic retinal tissues, embryonic stem cells, mesenchymal stem cells, induced pluripotent stem cells (iPSCs), or iPSC-derived retinal organoids. In this and other embodiments, the purified population of cells are derived from embryonic retinas. In certain embodiments, the purified population of cells comprise at least 50% of a total number of cells in the composition having an expression marker profile of CD73⁺, Thrb⁺, CD11b⁻. In certain embodiments, the purified population of cells comprise at least 75% of a total number of cells in the composition having an expression marker profile of CD73⁺, Thrb⁺, CD11b⁻. In certain embodiments, the purified population of cells comprise at least 75% of a total number of cells in the composition having an expression marker profile of CD73⁺, Thrb⁺, CD11b⁻. In certain embodiments, the purified population of cells comprise at least 85% of a total number of cells in the composition having an expression marker profile of CD73⁺, Thrb⁺, CD11b⁻. In certain embodiments, the purified population of cells comprise at least 90% of a total number of cells in the composition having an expression marker profile of CD73⁺, Thrb⁺, CD11b⁻. In certain embodiments, the purified population of cells comprise at least 95% (or more) of a total number of cells in the composition having an expression marker profile of CD73⁺, Thrb⁺, CD11b⁻. CD11b⁻marker indicates the lack of microglia and other cells which express CD11b, e.g. monocytes, granulocytes, macrophages NK cells etc., in the purified cell population, thereby, the cells in the composition are not immunogenic. In these and other embodiments, an isolated cell having an expression marker profile of CD73⁺, Thrb⁺, CD11b⁻comprises a vector expressing a polypeptide, an exogenous nucleic acid sequence, polynucleotides, oligonucleotides, polypeptides, peptides or combinations thereof.

Other markers for cone photoreceptor cells, include without limitation: RXRG (retinoid X receptor (RXR) gamma), THRB (thyroid hormone receptor beta), SALL3 (Sall-like protein 3), ONECUT1 (One cut domain, family member 1), OPN1SW (Opsin 1 short wavelength), OPN 1 LW/MW (Opsin 1 long wavelength and medium wavelength), ARR3 (Arrestin -C-3), GNAT2 (G-protein subunit alpha transducing 1), CNGB3 (cyclic nucleotide-gated (CNG) channel beta subunit), PDE6H (phosphodiesterase 6H), PDE6C (phosphodiesterase 6C), GUCA1A (guanylate cyclase activator 1A).

Accordingly, in these and other embodiments, a cone photoreceptor cell comprises one or more markers comprising: RXRG, THRB, SALL3, ONECUT1, OPN1SW, OPN 1 LW/MW, ARR3, GNAT2, CNGB3, PDE6H, PDE6C, GUCA1A or combinations thereof. In certain embodiments, an early cone photoreceptor cell comprises one or more markers comprising: RXRG, THRB, SALL3, ONECUT1 or combinations thereof. The markers: RXRG, THRB, SALL3, ONECUT1 are markers for early cone photoreceptor. The markers: OPN1SW, OPN 1 LW/MW, ARR3, GNAT2, CNGB3, PDE6H, PDE6C, GUCA1A are markers for mature cone photoreceptor cells.

In certain embodiments, a method of producing progenitor photoreceptor cells, comprises culturing retinal progenitor cells; isolating CD73⁺cells from the cultured retinal progenitor cells; culturing the CD73⁺cells; subjecting the CD73⁺cells to a second isolation step comprising isolating CD73⁺Thrb⁺ and CD11b⁻cells; culturing and expanding the CD73+Thrb⁺CD11b⁻cells; to produce the progenitor photoreceptor cells. In these and other embodiments, the retinal progenitor cells are derived from embryonic retinas, embryonic stem cells, mesenchymal stem cells, induced pluripotent stem cells (iPSCs), or iPSC-derived retinal organoids. In certain embodiments, the retinal progenitor cells are derived from embryonic retinas or embryonic retinal tissues. In certain embodiments, the embryonic retinas or retinal fetal tissues are contacted with an enzyme to obtain a cell suspension. In these and other embodiments, the CD73⁺Thrb⁺CD 11 b⁻cells comprise at least 90% of the total cell counts. In these and other embodiments, the CD73⁺Thrb⁺CD11b⁻cells comprise at least 95% of the total cell counts.

In certain embodiments, a method of producing progenitor photoreceptor cells, comprises: obtaining embryonic retinas or retinal tissues and dissociating the embryonic retinas or retinal tissue with an enzyme to produce a cell suspension; culturing cells obtained from the cell suspension; isolating CD73⁺cells from the cell culture and further culturing CD73⁺cells; subjecting the CD73⁺cells to a second isolation step comprising isolating CD73⁺Thrb⁺ and CD 11 b⁻cells; culturing and expanding the CD73⁺Thrb⁺CD 11 b⁻cells, to produce the progenitor photoreceptor cells. In these and other embodiments, the progenitor photoreceptor cells can be cultured with one or more agents or culturing conditions to produce a desired mature cell phenotype. In these and other embodiments, the one or more agents comprise growth factors, cytokines, reprogramming factors, hormones, cells, tissues or combinations thereof. In certain embodiments, the culturing conditions comprise: culturing substrates, co-culturing environment, two- or three-dimensional culturing. In certain embodiments, progenitor photoreceptor cells are precursors of cone photoreceptor cells. In these and other embodiments, the cone photoreceptor cells identified by markers comprising: Cone Arrestin⁺, Red/G opsin⁺Rhodopsin⁻.

In certain embodiments, a method of producing a purified population of progenitor cells, comprises isolating cells from a biological sample; culturing and expanding the cells; isolating cells based on a first biomarker profile and further culturing of the isolated cells; subjecting the cultured isolated cells to a second isolation step based on a second biomarker profile; thereby, producing a purified population of progenitor cells. In these and other embodiments, the biological sample comprises: fetal tissues, embryonic tissues, extraembryonic, tissues, cord blood, fluids, cord tissues, bone marrow, adult tissues or combinations thereof.

In certain embodiments, a method of screening for a candidate therapeutic agent comprises contacting a cell embodied herein, with a candidate therapeutic agent; comparing genotypic and/or phenotypic characteristics and/or induction of differentiation of the cell to a baseline control in the presence or absence of the candidate therapeutic agent and correlate responses to specific genetic or phenotypic features. Typical assays known in the art can be used to identify genotypic or phenotypic changes such as, gene analysis, probes, gels, PCR, blots, enzyme assays, immunochemistry assays and the like. In these and other embodiments, the candidate agent is selected for its therapeutic properties, for example, neuroprotective, survival of cells, proliferation, does not kill the cells, induces the engraftment of the progenitors into the tissues, allows for differentiation into a desired cell-type, etc.

In certain embodiments, a method of treating an ocular or retinal disease comprises administering to a subject an effective amount of the isolated and purified population of progenitor cells embodied herein.

Other aspects are described infra.

### Definitions

The terminology used herein is for the purpose of describing particular embodiments only and is not intended to be limiting of the invention. Unless specifically defined otherwise, all technical and scientific terms used herein shall be taken to have the same meaning as commonly understood by one of ordinary skill in the art (e.g., in cell culture, molecular genetics, and biochemistry). It is understood that where a parameter range is provided, all integers within that range, and tenths thereof, are also provided by the invention. For example, “0.2-5 mg” is a disclosure of 0.2 mg, 0.3 mg, 0.4 mg, 0.5 mg, 0.6 mg etc. up to and including 5.0 mg.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the term “about” in the context of a numerical value or range means ±10% of the numerical value or range recited or claimed, unless the context requires a more limited range.

As used herein, the term “agent” or “candidate therapeutic agent” is meant to encompass any molecule, chemical entity, composition, drug, therapeutic agent, chemotherapeutic agent, or biological agent capable of preventing, ameliorating, or treating a disease or other medical condition. The term includes small molecule compounds, antisense reagents, siRNA reagents, antibodies, enzymes, peptides organic or inorganic molecules, natural or synthetic compounds and the like. An agent can be assayed in accordance with the methods of the disclosure at any stage during clinical trials, during pre-trial testing, or following FDA-approval.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

As used herein, “effective” when referring to an amount of a therapeutic compound refers to the quantity of the compound that is sufficient to yield a desired therapeutic response without undue adverse side effects (such as toxicity, irritation, or allergic response) commensurate with a reasonable benefit/risk ratio when used in the manner of this disclosure.

As used herein, a “purified” population of cell types, e.g. progenitor cone receptor cells are substantially free of other cell populations. In certain embodiments, the cell populations are based on a biomarker profile, that is presence and/or absence of certain biomarkers, in the cell population. Purified cell populations comprise at least 50% of a total number of cells in the composition having a certain expression marker profile, for example, an expression marker profile of CD73⁺, Thrb⁺, CD11b⁻. Preferably, the cell population is at least 75%, at least 90%, and at least 99%, pure. For example, a purified cell population is one that is at least 85%, 90%, 91%, 92%, 93%, 94%, 95%, 98%, 99%, or 100% (w/w) of the desired cell type. Purified also defines a degree of sterility that is safe for administration to a human subject, e.g., lacking infectious or toxic agents.

The terms “subject,” “patient,” “individual,” and the like as used herein are not intended to be limiting and can be generally interchanged. That is, an individual described as a “patient” does not necessarily have a given disease, but may be merely seeking medical advice.

The terms “therapeutically effective amount,” “treatment effective amount” and “effective amount” as used herein are synonymous unless otherwise indicated, and mean an amount of a compound, peptide or composition of the present invention that is sufficient to improve the condition, disease, or disorder being treated and/or achieved the desired benefit or goal (e.g., control of body weight). Those skilled in the art will appreciate that the therapeutic effects need not be complete or curative, as long as some benefit is provided to the subject.

As used herein, “treating” encompasses, e.g., inhibition, regression, or stasis of the progression of a disorder. Treating also encompasses the prevention or amelioration of any symptom or symptoms of the disorder. As used herein, “inhibition” of disease progression or a disease complication in a subject means preventing or reducing the disease progression and/or disease complication in the subject.

All genes, gene names, and gene products disclosed herein are intended to correspond to homologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates. Thus, for example, for the genes or gene products disclosed herein, which in some embodiments relate to mammalian nucleic acid and amino acid sequences, are intended to encompass homologous and/or orthologous genes and gene products from other animals including, but not limited to other mammals, fish, amphibians, reptiles, and birds. In preferred embodiments, the genes, nucleic acid sequences, amino acid sequences, peptides, polypeptides and proteins are human.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

All published foreign patents and patent applications cited herein are incorporated herein by reference. Genbank and NCBI submissions or other database references to protein sequence and/or nucleic acide sequences indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

### BRIEF DESCRIPTION OF THE DRAWINGS

FIG. 1 is a schematic illustration showing the differentiation and of multipotent retinal neuroepithelial cells (right hand panel). The top left hand panel is a photograph showing a cell sorter. The bottom left panel illustrates the markers of the cone precursor and cone cell.

FIG. 2 is a schematic illustration of the protocol used as described in the examples section.

FIG. 3 is a schematic illustration and a graph showing a summary of the protocol and the number of cells produced at each of the sorting steps. First sort (CD73⁺+). Second sort (CD73⁺/Thrb+/CD 11 b⁻). Microglia have been removed.

FIGS. 4A-4C are a series of scanned copies of photographs showing results from an experiment using bright field microscopy to isolate retina from embryonic eye tissue. FIG. 4A: First step is to cut the globe carefully to remove entire retina without a trace of RPE or surrounding ciliary layers. Dissociated retina was then digested using papain enzyme and single cell viability determined using Trypan blue. 90% and above viable cells are obtained by end of enzymatic digestion. FIG. 4B: Cells were cultured in fibronectin-coated t-75flask for 1 week. Upon reaching confluency cells were trypsin treated so as to obtain a single cell suspension. First sorting was performed using CD73 PE and APC surface markers. FIG. 4C: Post sorting CD73 positive cells (shown by the arrows) were cultured in fibronectin-coated flask containing media for retinal progenitor cells and knock-serum media in equal concentration. The bright-field image 24 hrs post sorting shows cell attachment, which reaches confluency in 2 weeks with viability of 92% on average.

FIGS. 5A-5H are a series of flow cytometry plots and tables showing the sorting strategy for cone precursor cells. Dissociated retina cultured for 1 week can reach 7-10 million cell count that is ideal for sorting. The first sort (FIGS. 5A-5D) is labeling the single cells with CD73 PE and APC surface marker. The input indicates the total number cells that went through the sorting step. CD73 positive cells in the input are about 40% cells that are enriched to 80% in the output chamber. Second sort (FIGS. 5E-5H) is more refined and strict in which double cone markers CD73 PE and thyroid receptor hormone B (Thrb) is used and CD11b (microgial marker) is used as dump channel. This is to ensure 100% deletion of microgial cells and by the end of second sort about 93% pure cone precursor cells (CD73+Thrb⁺CD 11⁻) is achieved.

FIGS. 6A and 6B are graphs showing the marker expression before and after sort. CD73 and Thrb expression were analyzed before and after each sort in order to confirm the checklist. After the first sort (FIG. 6A) an average of 80% expression of CD73 (arrow) was found and after the second sort (FIG. 6B) an average of 91% of both CD73 and Thrb expression (arrow) was found on the cells (N=4). One way ANOVA was performed and significant difference were found (*p<0.03, **p<0.0045).

FIGS. 7A and 7B are graphs showing the flow cytometry data pre and post sort. Different markers were analyzed using flow cytometry to evaluate the phenotype of the cells. Different conditions were analyzed ((after single sort (FIG. 7A), after second sort, 14 weeks, 16 weeks (FIG. 7B)). Cones expression markers (Arrestin (arrow), S-opsin and RG opsin) show higher levels after the second sort and reach 90% for both Arrestin and RG opsin. Rhodopsin (rod marker) is negative for all conditions. Recoverin (photoreceptor marker) shows an increase for cells coming from 16 weeks old fetus.

FIG. 8 is a series of fluorescent tissue stains demonstrating the characterization of the cells post sorting using immunocytochemistry to confirm the cell identity. Cone arrestin is multifunctional protein responsible for regulating and trafficking majority of G protein couple receptors. It is predominately expressed by the cone photoreceptor cells (green), R/G opsin (Green) marker of long and middle wavelength sensitive cone photoreceptor and S opsin (green) marker for short wavelength sensitive cone photoreceptor cells. Ki-67 (red) is used for identifying proliferating cells. Recoverin (green) is Ca+binding protein expressed by retinal photoreceptors and midget cone bipolar cells. Rod photoreceptor cells that mediate dim light specifically express rhodopsin (green). Cell nuclei are counterstained with DAPI (blue).

FIG. 9 is a graph and a table demonstrating the level and speed of cone cell integration into the retinas of host rats.

FIG. 10 is a series of fluorescent tissue stains showing the results from xenograft survival of CD73 double-sorted cells in the sub-retinal space of non-immunosuppressed Long Evan rats. STEM 121 (green) marker for human cells was used to locate the implanted cells. Cell nuclei of tissue were counterstained with DAPI (blue). Widespread distribution of implanted human cone precursor cells (green) extending processes towards the host photoreceptor layers can be observed. Cellular bodies co-localizing with DAPI indicates integration of implanted (green) cells with the host tissue at D3. Most of cells in the first row show DAPI co-staining indicating implanted cells integrated with the host tissue rather than transfer of cytoplasm.

FIG. 11 is a series of fluorescent tissue stains demonstrating the efficiency of the CD73 sorted cells to survive and integrate with the host retina. The study was performed for three days in a non-immunosuppressed long Evan rats. Stem 121 (green) marks human cells, cone arrestin (red) was used for photoreceptors and cell nuclei stained with DAPI. Human cone sorted cells (green) was mostly found in the photoreceptor layers on the host. The merged image shows co-localization (orange) of stem 121 (green) with cone arrestin (green) with distinct nuclei indicating the ability of these cells to migrate and integrate into the host retina.

FIG. 12 is a series of fluorescent tissue stains showing the results from xenograft study performed in Long Evan rats. Stem 121 (green) marker for human cells and cone arrestin (red) marker for cone photoreceptor was used to identify the survival and integration efficiency of CD73 double sorted cells in the sub-retinal space of non-immunosuppressed rats. The results shows clearly group of cells positively stained for human and cone marker in the outer nuclear layer of the host. Co-localization (yellow) indicates the ability of these cells to maintain its lineage (retain cone arrestin), migrate and integrate post-transplantation.

FIG. 13 is a series of fluorescent tissue stains showing results from the xenograft study. The Stem 121 (green) marker for human cells, cone arrestin (red) marker for cone photoreceptor and DAPI for cell nuclei was used for identification of transplanted cells. CD73 sorted cells (green) were found in different areas of host retina mostly concentrating in the photoreceptor layers. Some cells integrated at the outer segment layer and were found to extend processes towards the outer nuclear layer of the host retina.

FIG. 14 is a series of fluorescent tissue stains showing results from the in vivo study performed in the long-Evan rats for 3 days. Injected cells were stained with Stem 121 (green) marker for human cells and cone arrestin (red) marker for cone photoreceptor. Cell nuclei were counterstained with DAPI. Most of the implanted cells were found concentrated at the outer nuclear layer of the retina. Every stem 121 cells were found to be cone arrestin positive clearly showing the behavior and protein expression of these sorted cell was not significantly affected post-transplantation.

FIG. 15 are two plots showing two examples of flow cytometer analysis of human fetal retinal cells labeled with thyroid receptor hormone b (Thrb)-APC conjugated, clearly showed a distinct peak for brightly labeled cells. These cells were gated for single cell population and highest fluorescent cells were sorted to remove any background noise and cell debris. Although the antibody used is known to bind to a nuclear membrane bound receptor, providing evidence of recognition of a surface bound structure. Importantly, when used with CD73 and CD11 b antibodies, cone precursors at high purity and viability were generated.

FIG. 16 are plots demonstrating a pure cone precursor population (left hand panel) with high viability (around 80%; right hand panel). These qualities have not been possible to obtain prior to this invention.

FIG. 17 is a graph showing the results from the isolation and purification process performed with two distinct sorts which give rise to 40 million hCP after 10 weeks of culture total number of cells throughout the process of sorting. Total number of cells in function of time (weeks of culture). Number of cells was measured in T75 flasks using Trypan blue and hematocytometer. The average number of cells after digestion of retina is 2 million and is cultured until reaching 10 million cells. Upon reaching this number cells are sorted; therefore, total number drops down. First sorted cells are cultured again until reaching 10 million. Then the second sort isolates cone progenitor cells which are cultured until reaching high number (40 million) of cells at 10 weeks.

FIG. 18 is a graph showing the number of cells post sorting process for different tissue ages. High number of cells (60-80 million) can be obtained independently of original tissue age after 1 month. Number of cells in different tissue (at 10, 14 and 16 weeks) was measured at each passage after the entire sorting process. The number of cells for each tissue start low (around 250,000) after the second and precise sort. Cells quickly start proliferating showing an exponential expansion and reaching 50 million cells for each tissue after 1 month. This provides evidence of the enhancing the proliferation and culture in order to reach higher cell numbers and create significant bank of cells.

FIG. 19 is a graph showing that the CD73 purification performed with the 1st sort increases from 20% to 80% (arrow). Data is shown as mean+SD for 10 replicates (10 different sorts). FIG. 19 shows the expression of sorting markers (CD73 and Thrb) during the sorting process for the input, output and negative fraction of cells. Data was analyzed using a cell sorter and flow cytometer. (Data is shown as mean+SD for 10 replicates (10 different sorts). Statistical one-way ANOVA was performed and shows significant difference between input and output (*p<0.001).

FIG. 20 is a graph showing that the CD73/Thrb purification performed with 2nd sort increases from 55% to 92% (arrow). Data is shown as mean+SD for 10 replicates (10 different sorts). Statistical one-way ANOVA was performed and shows significant difference between input and output (*p<0.001).

FIG. 21 is a graph showing that Thrb alone purification performed with 2nd sort increases from 45% to 96% (arrow). Data is shown as mean+SD for 10 replicates (10 different sorts). Statistical one-way ANOVA was performed and shows significant difference between input and output (*p<0.001).

FIG. 22 is a series of plots, histograms and a table showing that the 1st sort allows for purification of CD73 positive cells -Clear staining in APC and PE from input to output and negative. The raw sorting markers expression analyzed with live flow cytometry. Expression of sorting markers (CD73, Thrb) with their specific fluorophore (APC, PE, VioBlue) before and after sort (arrows). Top left panel: scatter plot of double CD73 expression for input, output and negative. Right panel: histogram of size of cells for all samples and of markers expression in APC. Bottom left panel: gating strategy and population number corresponding to the gates. Histogram in both figures shows that, post sorting, CD73 and Thrb positive cell population was increase (arrows). This is proven by the higher intensity of the peaks of these markers. A minimum of 50,000 cells was used for each sample in flow cytometry.

FIG. 23 is a series of plots, histograms and a table showing that the 2nd sort allows for purification of CD73 and Thrb positive cells —Clear population in the input and output section. Top left panel: scatter plot of double CD73 expression for input, output and negative. Right panel: histogram of size of cells for all samples and of markers expression in APC. Bottom left panel: gating strategy and population number corresponding to the gates. Histogram in both figures shows that, post sorting, CD73 and Thrb positive cell population was increased (arrows). This is proven by the higher intensity of the peaks of these markers (arrows). A minimum of 50,000 cells was used for each sample in flow cytometry.

FIG. 24 is a graph showing that recoverin is constant, Calbidin is increasing(arrow), Rhodopsin is negative through the 2 sorts. Expression of cone markers (cone Arrestin, Blue Opsin, RG Opsin), RGC makers (RBPMS, Brn3a), bipolar cells (PKCa), rod (Rhodopsin), photoreceptors (Recoverin) and amacrine cells (Calbindin) were analyzed using the MACSQuant flow cytometer. FIG. 24: rods, photoreceptors and amacrine cells markers. Expression of markers was measured before the 1st sort, during both sorts and after the 2nd sort.

FIG. 25 is a graph showing the results from RBPMS and Brn3a positive cells are sorted out in the first sort. Half of PKCa cells (arrow) are sorted out in the first sort demonstrating the presence. of bipolar and ganglion cells markers. Expression of markers was measured before the 1st sort, during both sorts and after the 2nd sort. Cone markers expression seems to stay constant during the first sort but rises quickly after the second sort, reaching almost 99%. This provides evidence that the second sort aims at targeting the final hCP population. Bipolar and ganglion cell markers expression reduce drastically after the first sort but stay constant during the second sort. The first sort aims at sorting out non-cone cells. Other markers are not affected by the sorts. A minimum of 50,000 cells was used for each sample in flow cytometry. Data is shown as mean+SD for 5 replicates (5 different flow experiments).

FIG. 26 is a graph showing that Cone Arrestin, Blue opsin and RG opsin positive cells were isolated in the second sort. These are cone cells markers. Expression of markers was measured before the 1st sort, during both sorts and after the 2nd sort. Cone markers expression seems to stay constant during the first sort but rises quickly after the second sort, reaching almost 99% (arrow). This provides evidence that the second sort aims at targeting the final hCP population. Bipolar and ganglion cell markers expression reduce drastically after the first sort but stay constant during the second sort. The first sort aims at sorting out non-cone cells. Other markers are not affected by the sorts. A minimum of 50,000 cells was used for each sample in flow cytometry. Data is shown as mean+SD for 5 replicates (5 different flow experiments).

FIG. 27 is a graph showing that only S-opsin and Recoverin are higher in 16 weeks sorted tissue compared to 14 weeks. Expression of cone markers (cone Arrestin, red/green Opsin, s-opsin), proliferation maker (ki67), od (Rhodopsin) and photoreceptors (Recoverin) were analyzed using flow cytometry. FIG. 27 shows the differences in all these markers between a double sorted 14 weeks tissue and 16 weeks tissue. Only s-opsin and recoverin expression show a high significant difference between 14 weeks and 16 weeks (being higher for the latest). Cone Arrestin and RG opsin expression reaches 98% after the second sort for 14 weeks tissue. Furthermore, these markers are low in the negative population. This provides evidence that the second sort is isolating and purifying cells with high expression of cone markers. A minimum of 50,000 cells was used for each sample in flow cytometry.

FIG. 28 is a graph showing that Cone Arrestin and RG opsin reach 98% after 2nd sort. This figure shows the evolution of these markers during the process of sorting for a 14 weeks tissue.

FIG. 29 is a graph showing that Cone Arrestin and RG opsin are high in sorted population and low in negative. This figure shows the results of cone markers expression for 14 weeks tissue in the input, negative fraction and output.

FIGS. 30A-30C are a series of plots showing the raw data of markers expression analyzed with fix flow cytometry. Expression of cone markers (cone Arrestin, Blue Opsin, RG Opsin), RGC makers (RBPMS, Brn3a), bipolar cells (PKCa), rod (Rhodopsin), photoreceptors (Recoverin) and amacrine cells (Calbindin) were analyzed using the MACSQuant flow cytometer. The first 9 panels show the expression for unsorted cells. The second 9 panels show the expression for first sorted cells and the last 9 panels show the expression for double sorted cells. Independent of the markers, a clear positive or negative population was seen and therefore was gated and analyzed. Cones markers are gradually increasing from the unsorted population to the last double sorted population while rods and RGC markers are decreasing, being really low at the end of the second sort. Only bipolar cells markers remain relatively present at the end of the process, suggesting that the only other population of cells present, aside from cone progenitors, are bipolar progenitor cells. A minimum of 50,000 cells was used for each sample in flow cytometry.

FIG. 31 is a series of immunostains showing a high positive number of cells expressing Cone Arrestin (arrows). The immunostaining analyses was performed on double sorted purified hCP after 3 weeks in culture. Cone Arrestin, Blue Opsin and S-opsin are distinct cone photoreceptors and hCP were stained with same primary antibodies. Cone high expression of cone Arrestin positive cells could be noted (arrows). Arrestin-C or Arr3 is multifunctional protein that controls signaling and trafficking of majority of G protein coupled receptors. This is mostly found in inner and outer segments along with inner plexiform layer of the retina. It is mainly expressed by cone photoreceptor and contributes to the shut-off mechanisms associated with high acuity of color vision. Three different hCP tested shows high expression of cone Arrestin protein confirming the linage of double-sorted cells. The nuclear stain used to identify the cells was DAPI.

FIG. 32 is a series of immunostains showing the high positive number of cells expressing Blue opsin (arrows). Blue opsin staining is second confirmative staining for the cone photoreceptors as full range of color perception is due to presence and functioning of different cone photoreceptors. Each type of cone cells possesses photo sensitive pigment protein that consist of cis-11 retinal and very unique opsin protein. Depending upon the sensitivity to the light peak they are classified as short wavelength (S-cone which has peak sensitivity around 420 nm), Long (L-cone with peak sensitivity at 560 nm) and middle range (M cone with peak sensitivity of 530 nm). S cone also known as blue cone or blue opsin is highly expressed in the double-sorted purified hCP population confirming the identity of isolated and purified cells. The nuclear stain used to identify the cells was DAPI.

FIGS. 33 and 34 are a series of immune staining for different markers for double sorted cells. Middle and long wavelength cone are stained with red/green (OPN 1 LW) is one of the highly conversed protein and most of the human cone photoreceptors is dominated by long and middle range cone cells. hCP cells were found to express all the cone photoreceptors specific protein providing clear indication of cell linage and its phenotypic expression. FIG. 33: Staining shows some Calbidin and RxRy positive cells (arrows). RxRy is one of the proteins expressed by post-mitotic cone cells and usually down-regulated at time S-opsin onset is noted. Few hCP cells were found positive of RxRy protein however, this express was significantly lower in-comparison with other cone photoreceptors.

FIG. 34 is a series of immune staining showing no Rhodopsin, some PKCa (arrow) and high Recoverin (arrow). Other retinal markers like Calbindin (Amacrine cells), Recoverin (photoreceptors), PKCa (Bipolar) and Rhodopsin (rod photoreceptors) were tested and expression varied from low to no expression. Nuclei were stained with DAPI and all images are taken at 20× magnification.

FIG. 35 is a graph showing the relative normalized expression of down regulation of pluripotent genes in hCP (50X4, CARDIO and MYCBP) with PCR. Total RNA quantification was performed using Qiagen RNA isolation kit. Total RNA extracted from hCP were used to synthesize cDNA with was further used for Q-RT-PCR. Gene tested were for pluripotency (Sox4, Card10, nanog, cMyc), early eye field (LHX2, PAX6). As the source of the hCP was fetal tissue it is critical to identify if the cells still express pluripotent markers which could cause tumor upon in vivo injection. The relative mRNA expression was normalized with housekeeping genes like Actin and GAPDH and 2 M method was used for calculation. The Q-RT-PCR data shows down regulation of pluripotent genes in hCP (SOX4, CARD10 and MYCBP). Cells used for the experiment were double sorted and cultured for 3 weeks. The control sample is unsorted 15-week fetal retina. Graph shows relative normalized expression.

FIGS. 36-38 are a series of immunostains of retinal sections of 2 week transplantation study. FIG. 36: In vivo transplantation in rats showed high engraftment of human-cone Arrestin positive cells in ONL (arrows). FIG. 37: The two weeks in vivo transplantation in rats showed colocalization of human and cone marker in transplanted cells (arrows). FIG. 38: 2 weeks in vivo transplantation in rats shows hCP in different layers positive for RG opsin confirming cone positive lineage (arrows). Survival of hCP donor cells in the rat retina was tested by injecting cells as suspension in sub-retinal space of Long Evans rats. After 2 weeks post injection, sections were stained with TRA 1-85 (human marker) and cone Arrestin (photoreceptor marker). TRA 1-85 positive cells co-localizing with cone Arrestin is noted after 14 days post transplantation. Donor cells were often seen aligning with host ONL or remain with INL cells. In the ONL, every TRA 1-85 positive cells was positive for cone Arrestin suggesting ability of hCP to survive, migrate and integrate with the host retina in a xenograft model. Another human marker used was Stem 12 along with another photoreceptor markers like R/G opsin. Similar to TRA 1-85 staining Stem 121 clearly shows ability of hCP to integrate with the host tissue along with capacity to retain its cone expression. Most of the human cells were found positive for cone photoreceptor markers. Abbreviation: GCL-ganglion cell layer, INL—inner nuclear layer, ONL—outer nuclear layer. Nuclei were stained with DAPI and all images are taken at 20× magnification. Scale bar—50 μm.

FIG. 39 is a series of plots showing the response to light with electrical activity proves that hCPCs are true active cones (arrow). These data show that hCPs are capable of responding to light stimulation, and therefore are true cones with this vital functional characteristic.

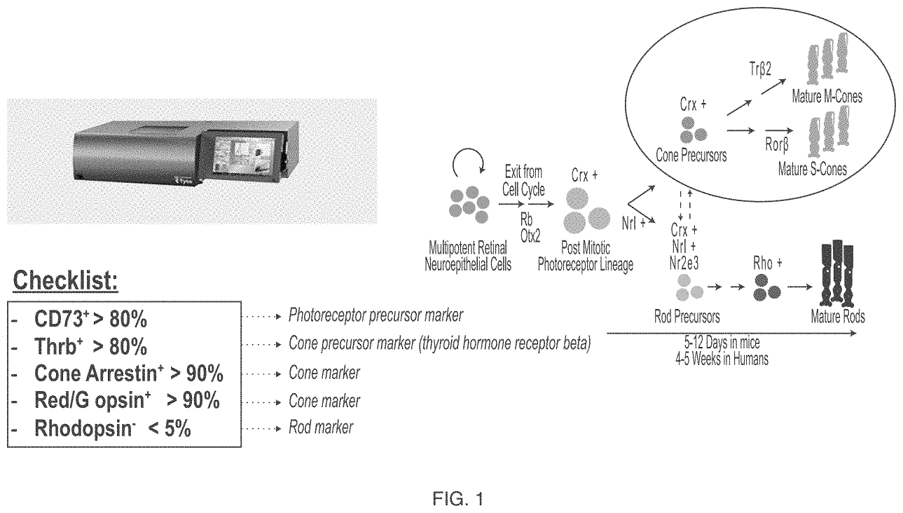

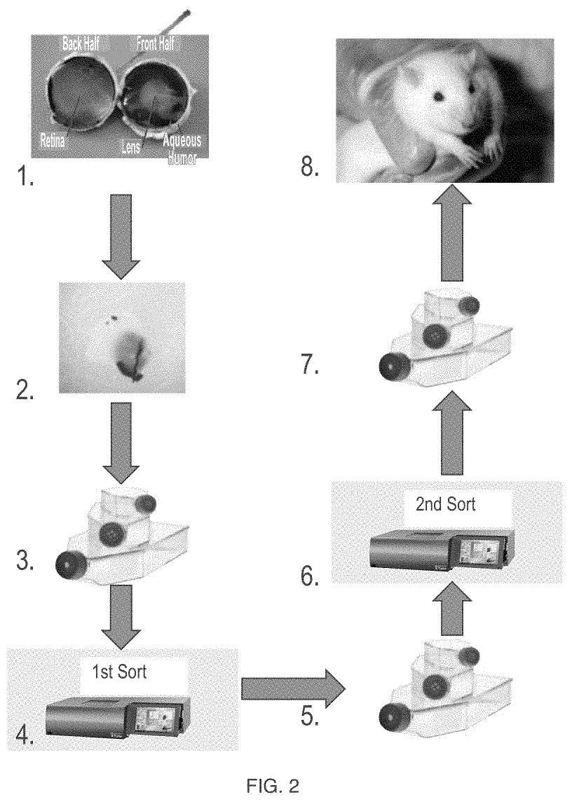

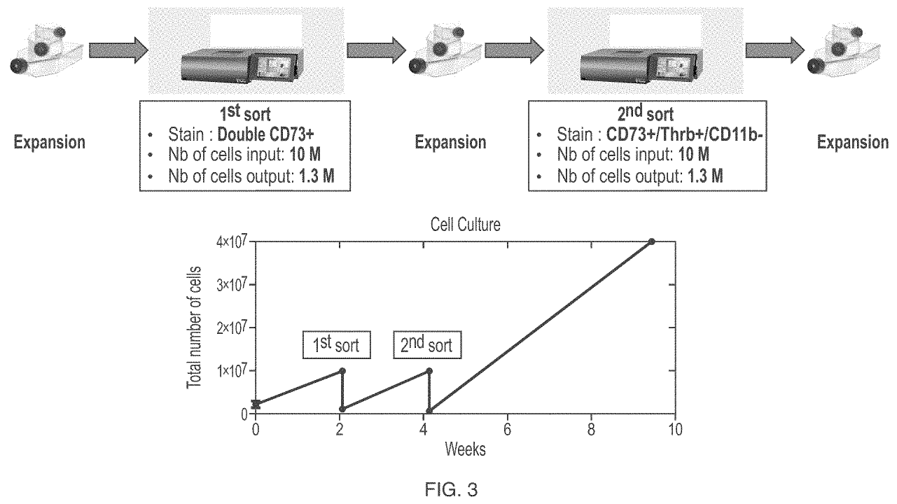

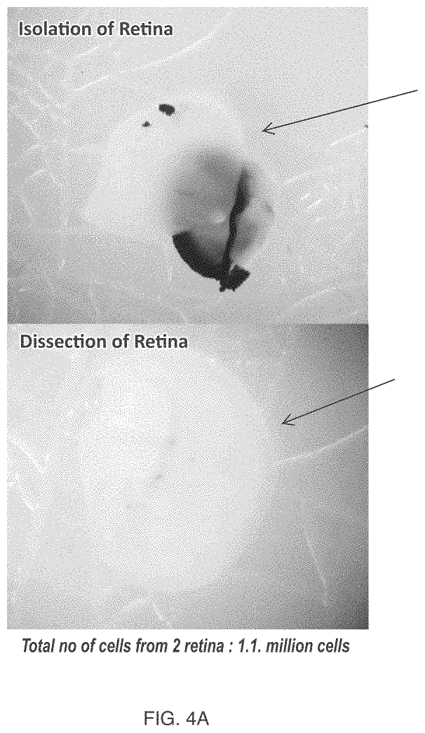

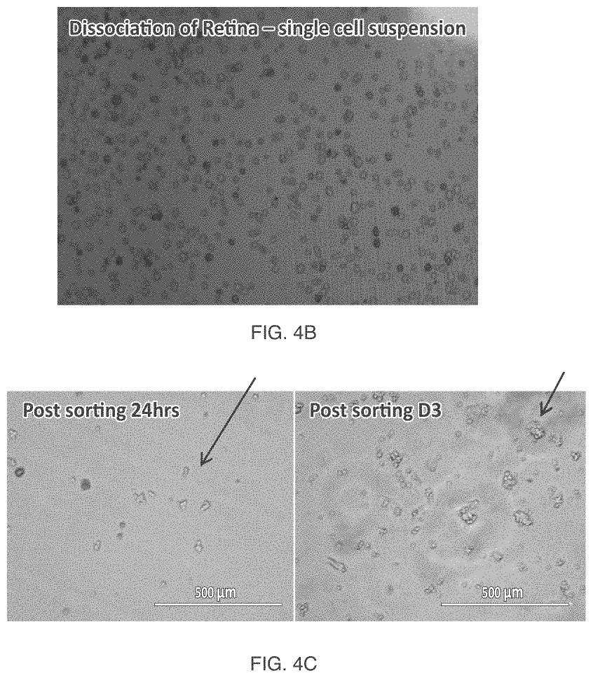

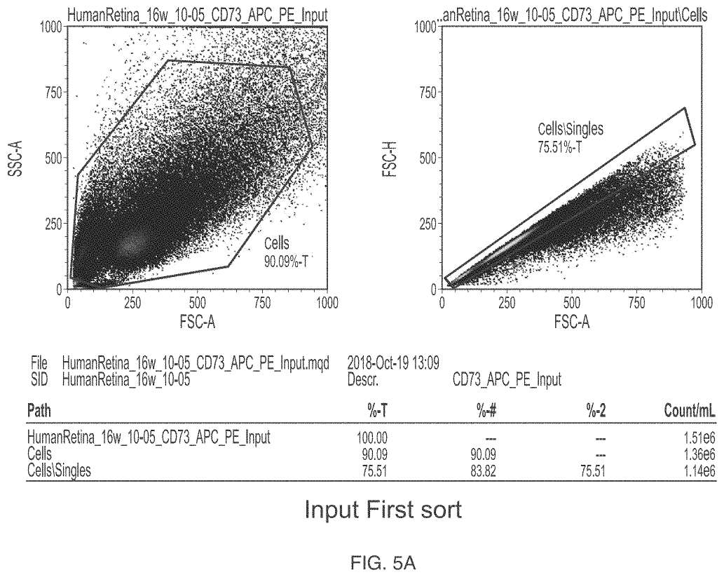

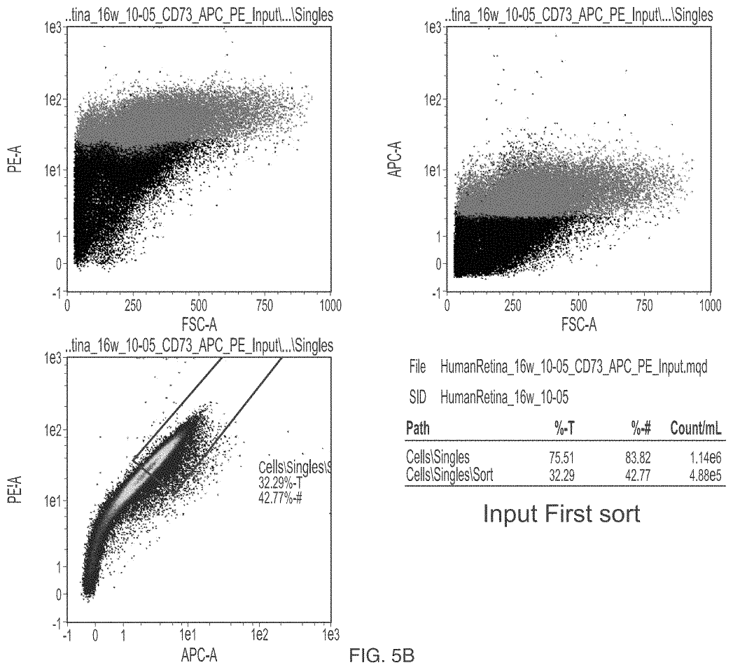

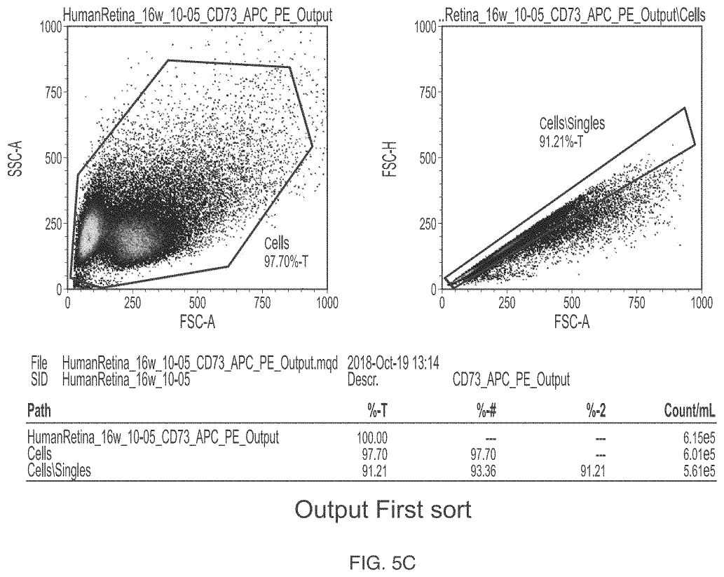

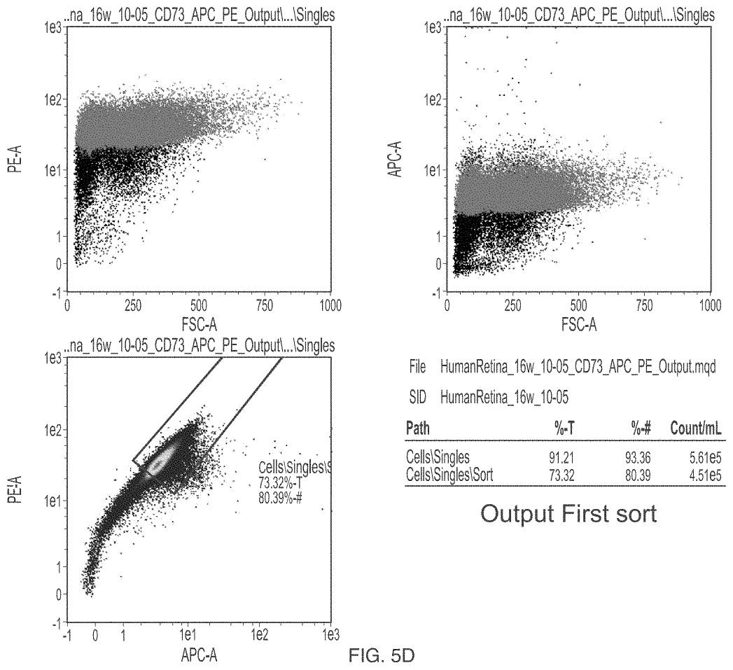

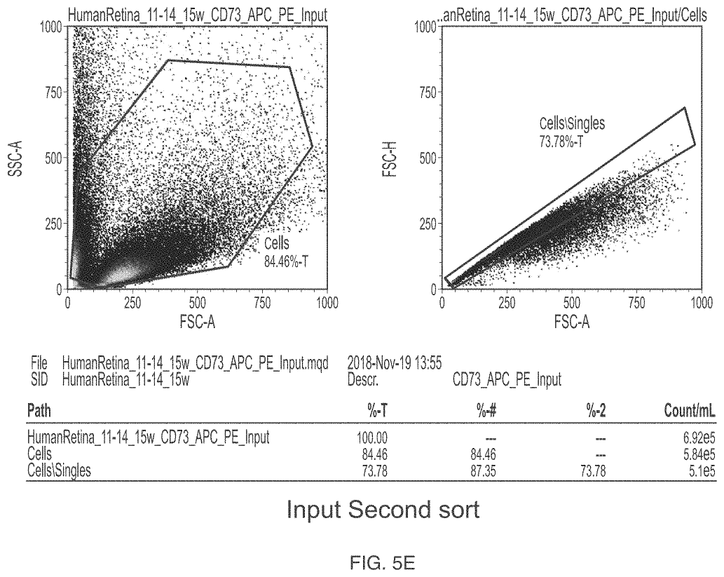

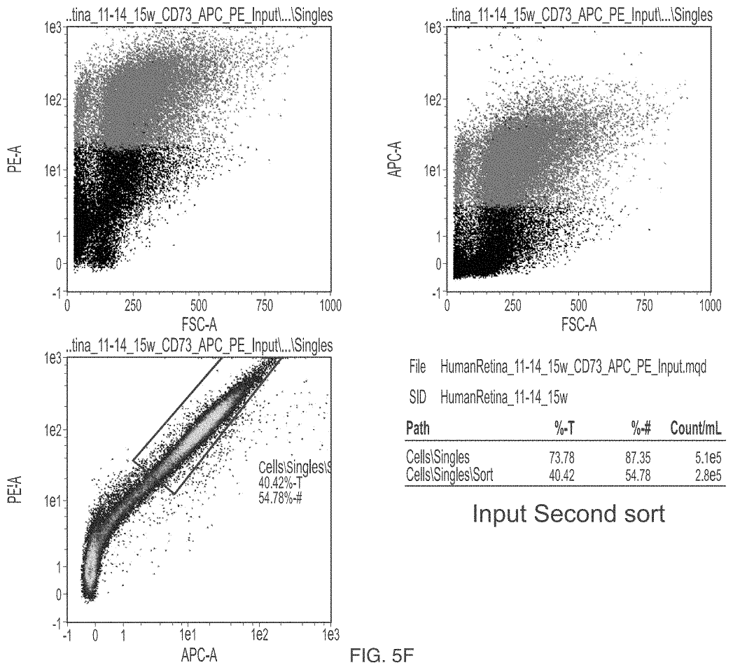

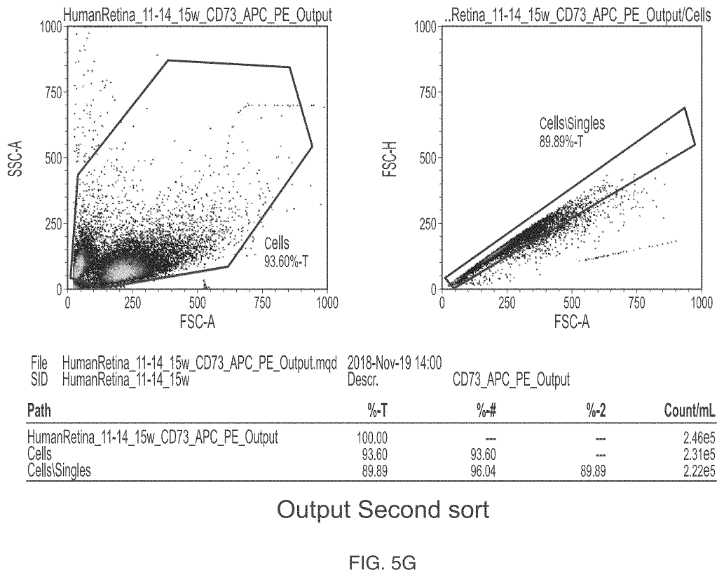

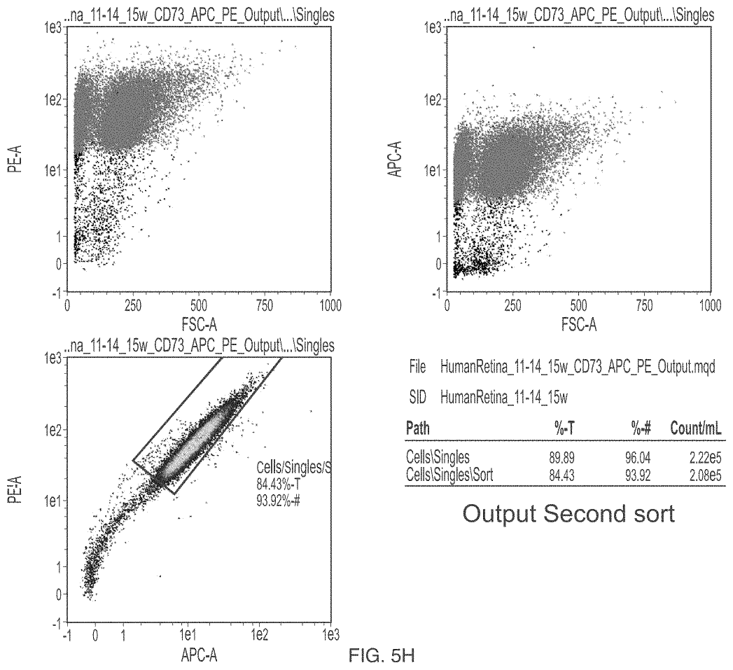

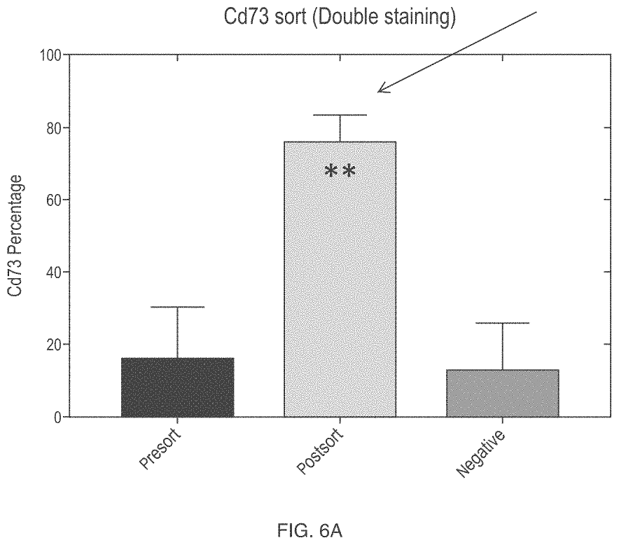

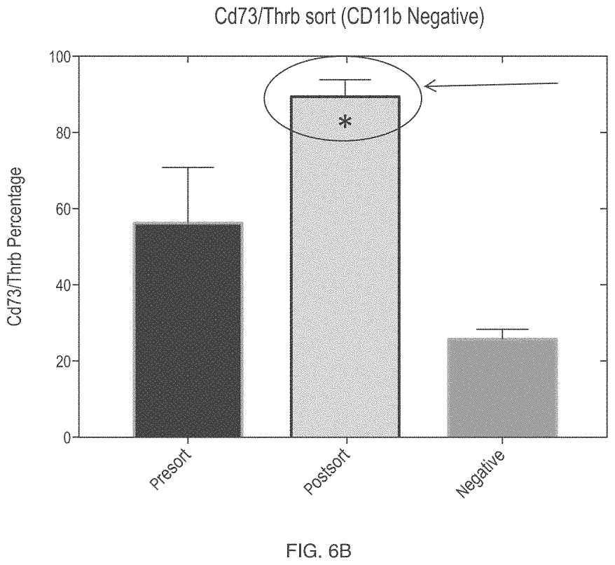

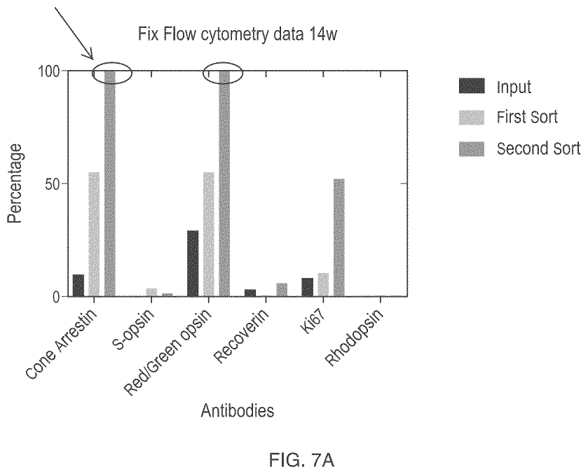

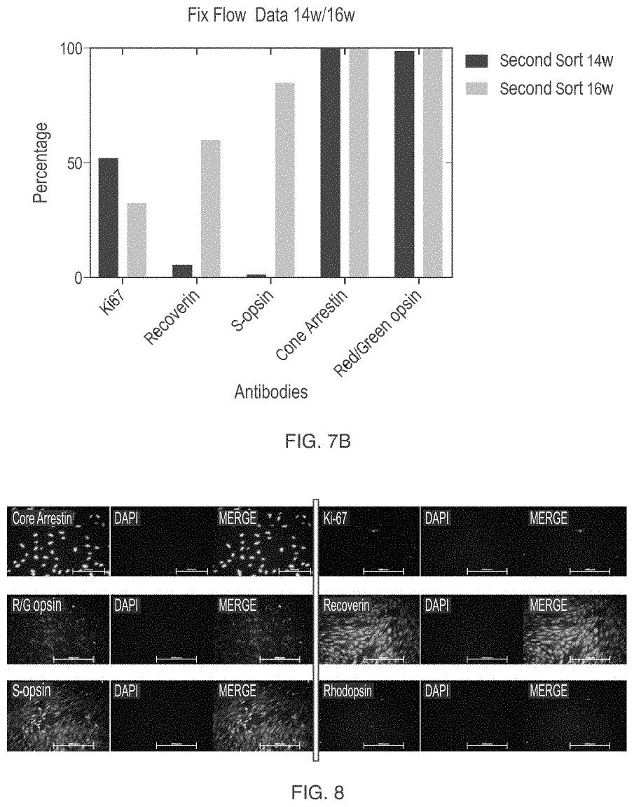

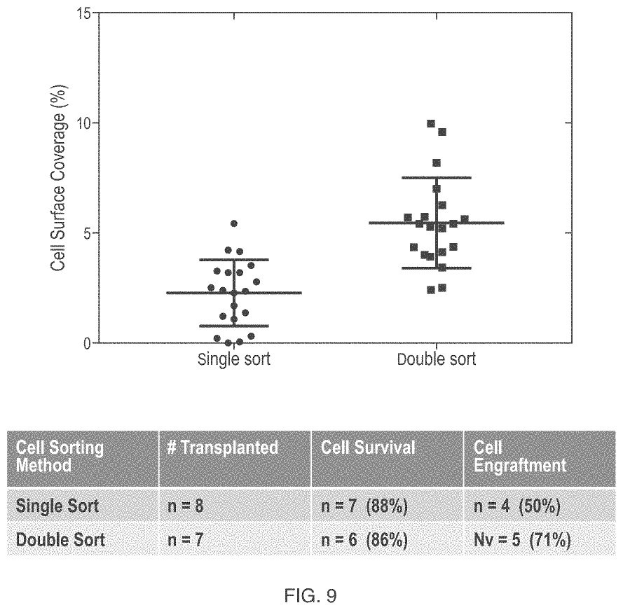

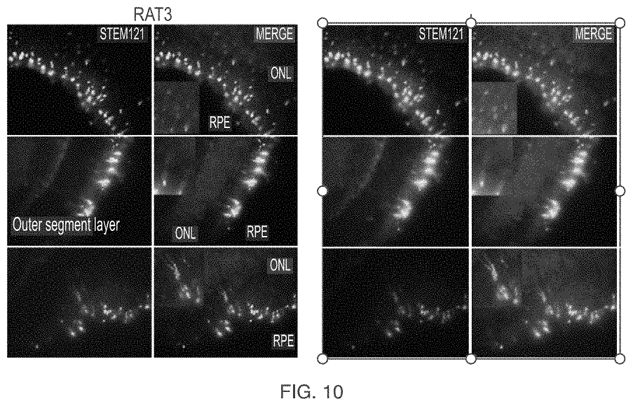

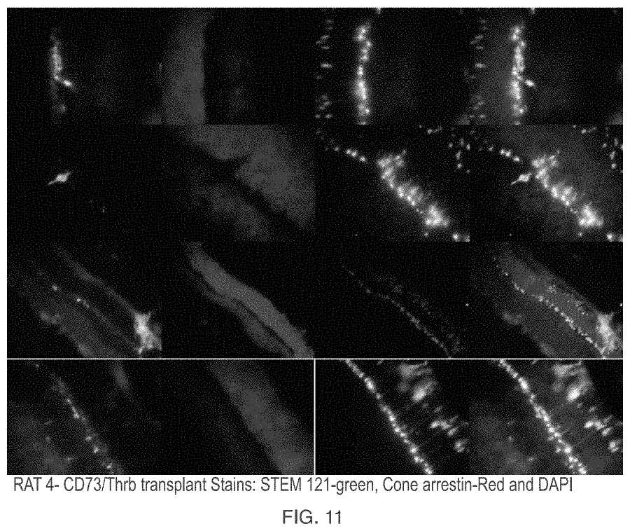

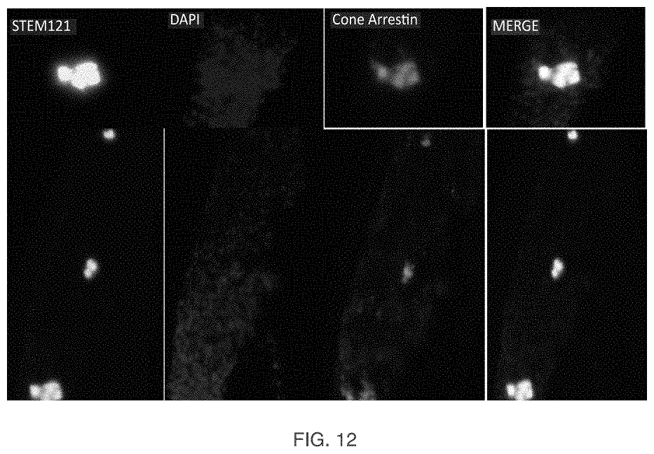

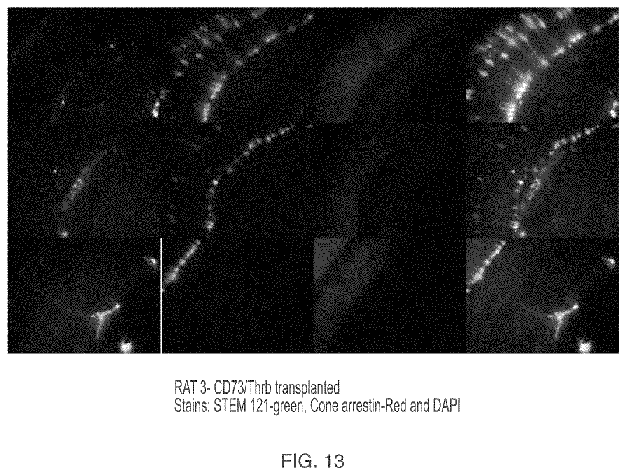

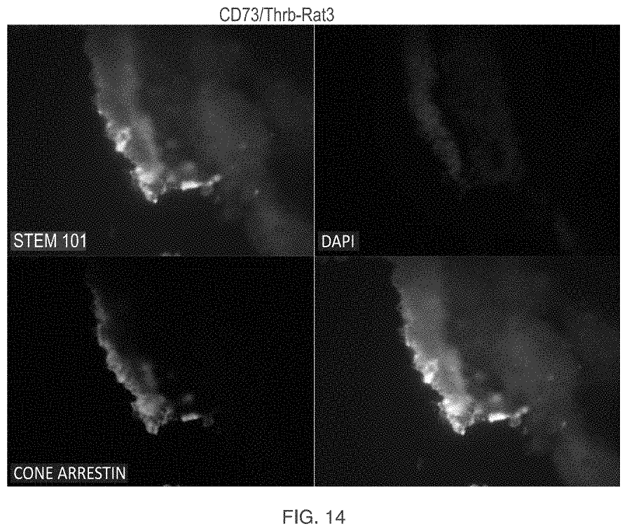

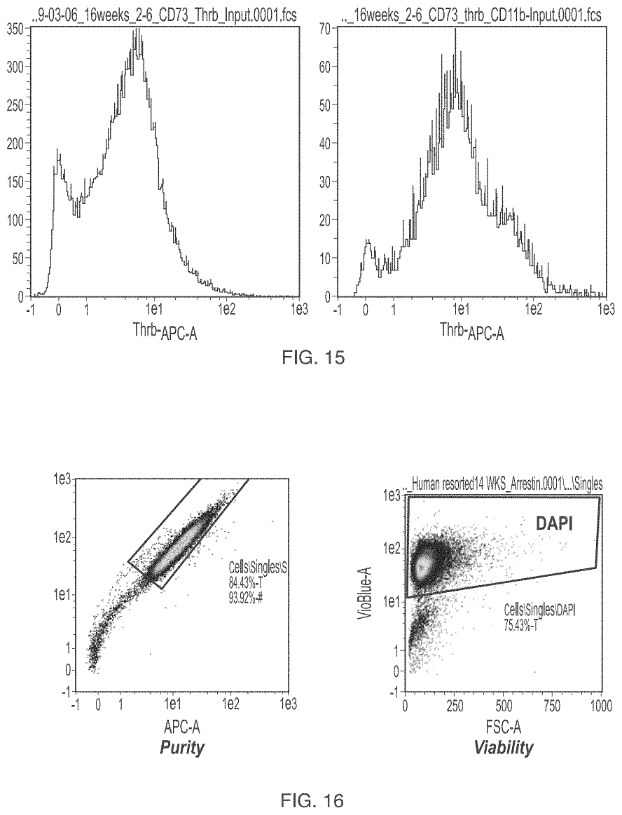

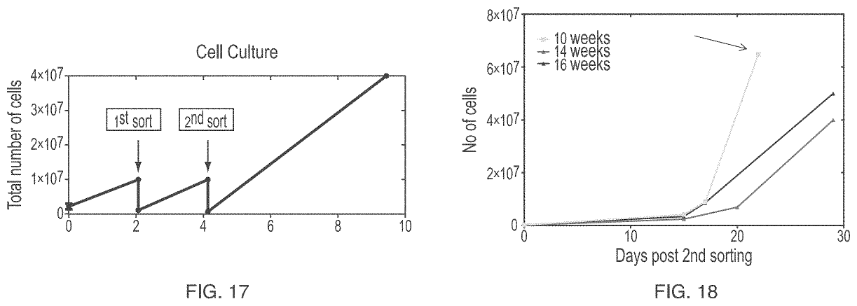

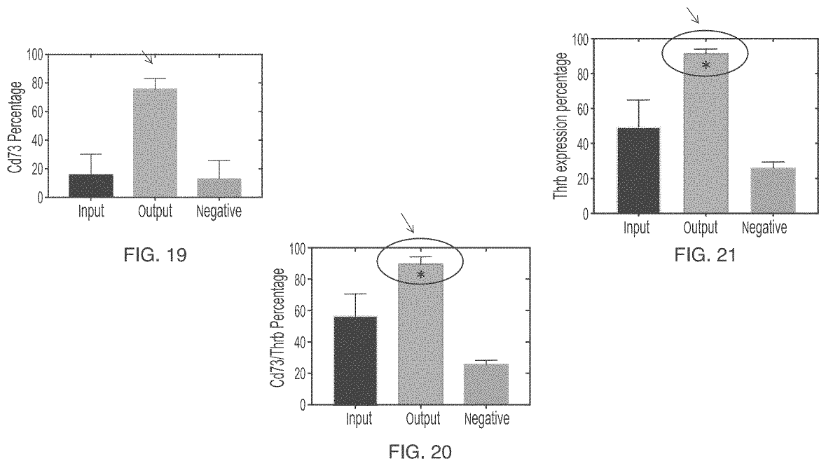

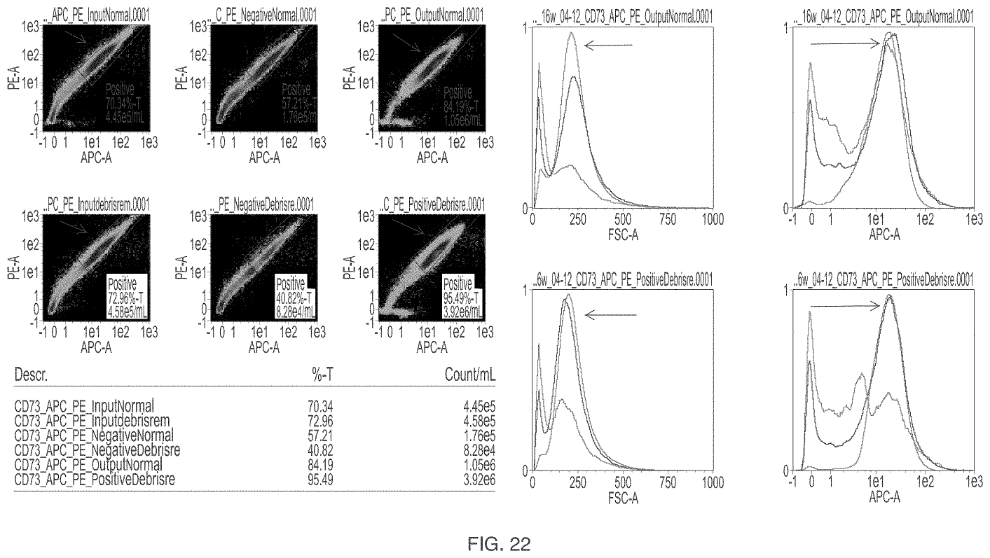

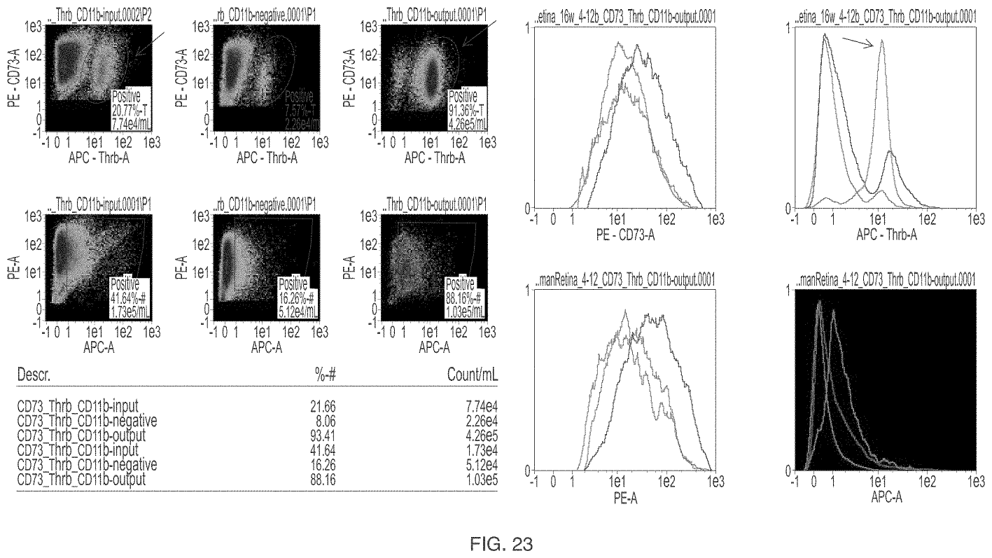

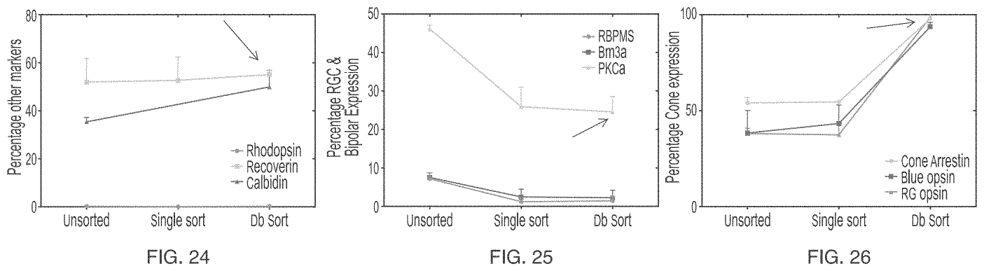

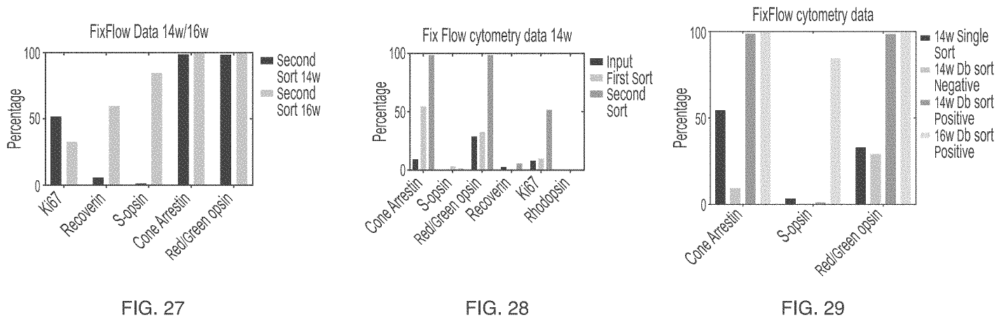

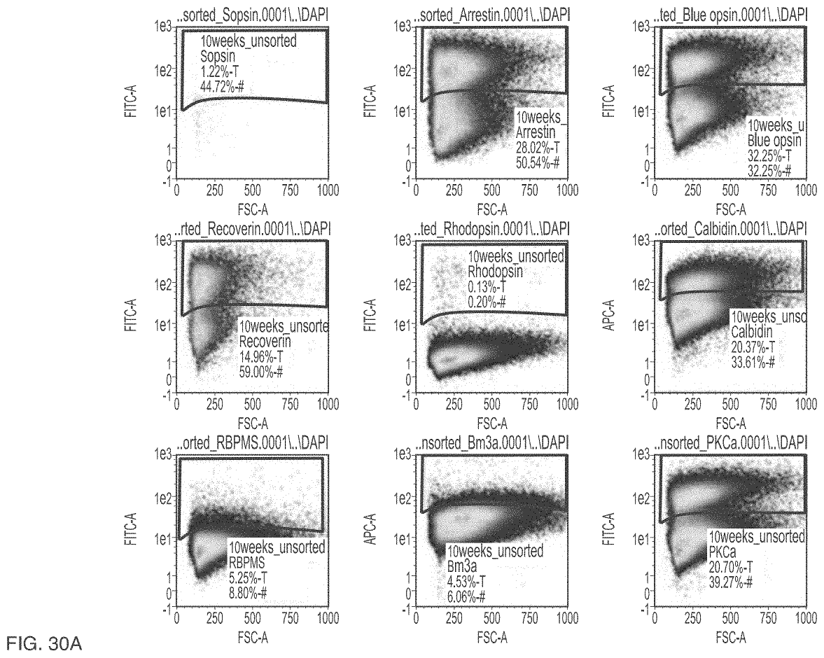

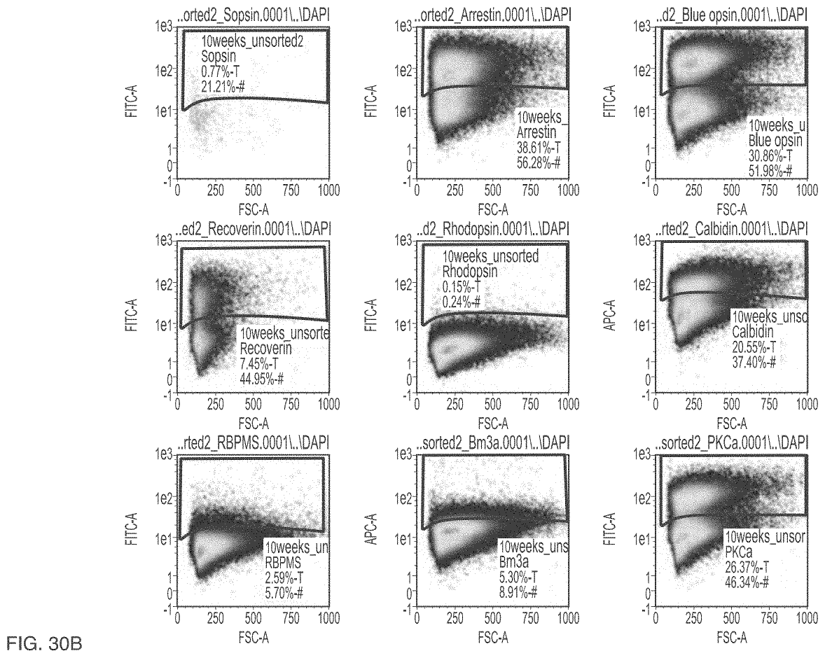

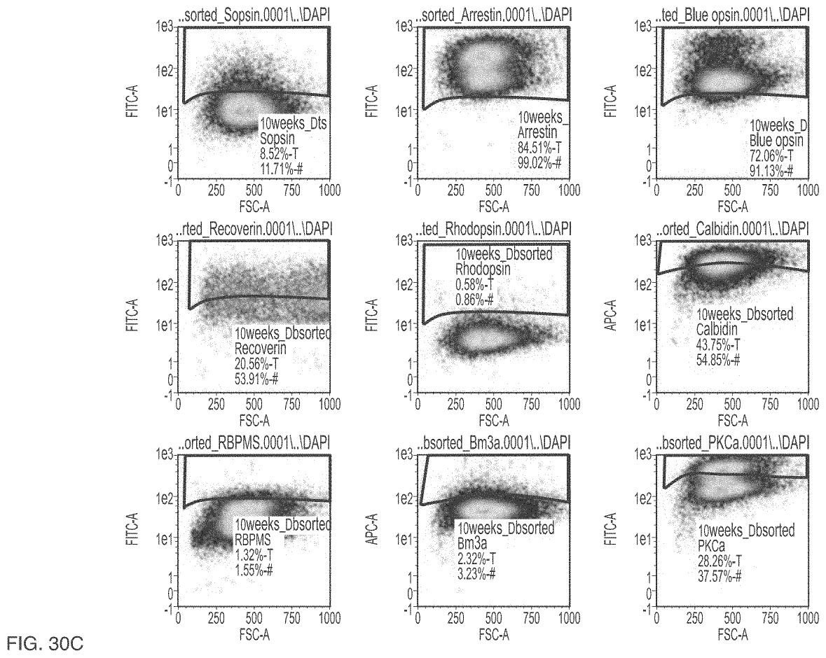

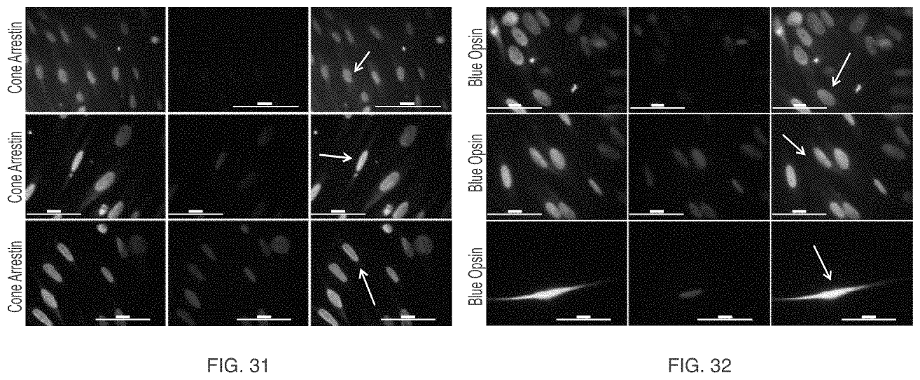

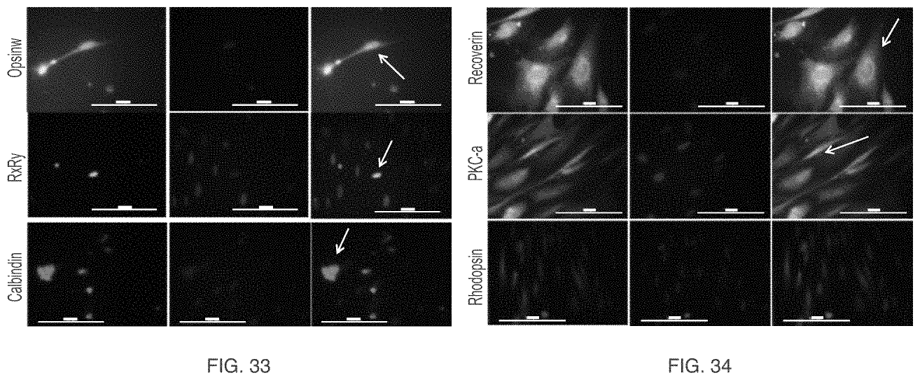

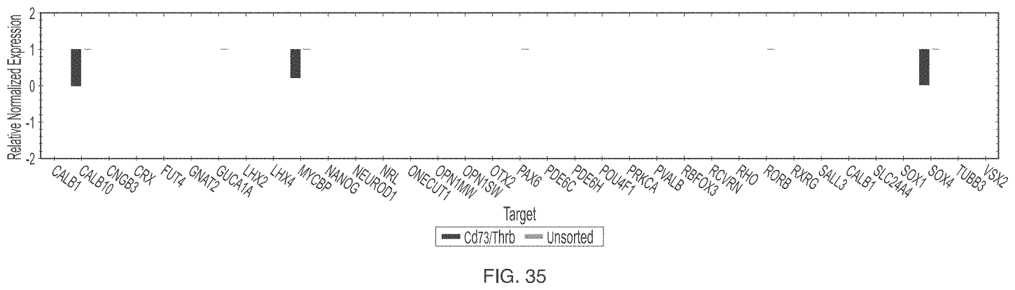

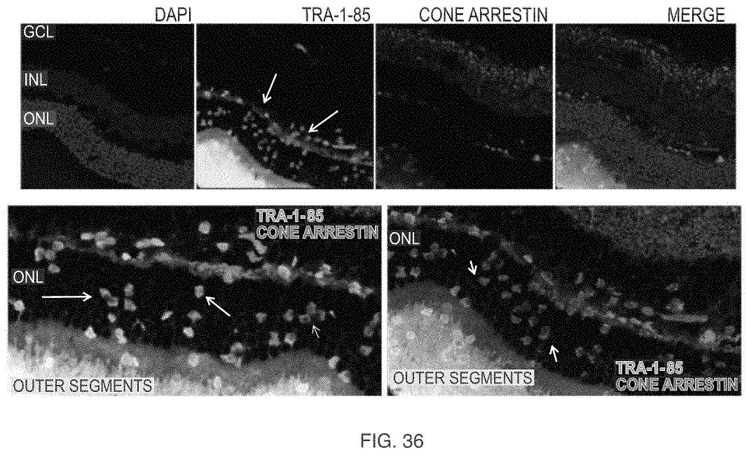

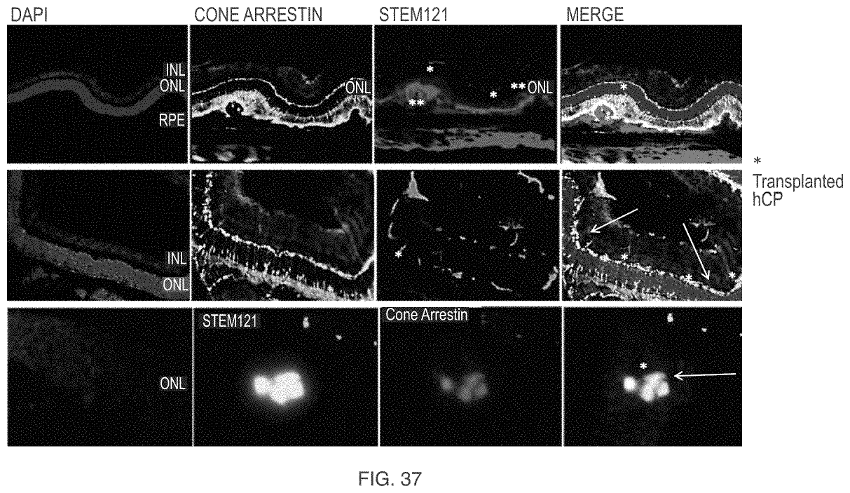

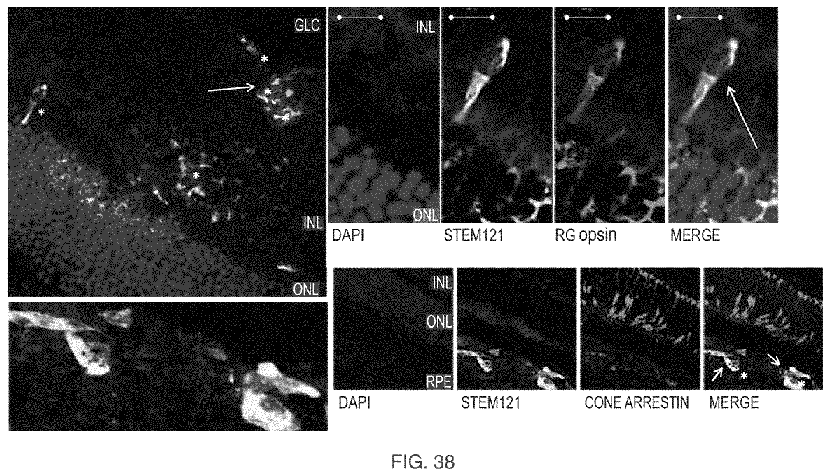

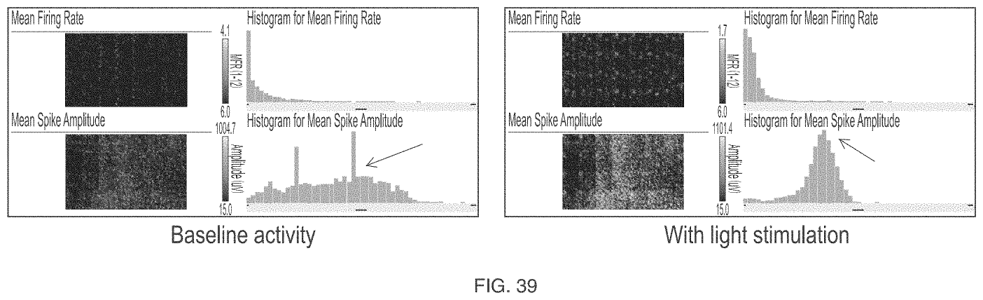

### DETAILED DESCRIPTION

ThrB was identified, herein, as a surface marker of cone progenitor cells. This is the first time ThrB has been used as a surface marker for labeling cells that were previously impossible to identify. Thrb is a transcription factor that can be released from the membrane into the nuclei or vice versa. The studies, herein, explored the time during which Thrb can be expressed on the cell surface and use it for labeling the cone progenitor cells (ThrB is exclusively expressed only by cone progenitor cells). The technique to use a microfluidic device for isolation of cone progenitor cells also enabled the enrichment of this rare population of cells with the highest purity and viability that has been reported. The microfluidic method allowed for labeling cells with both positive (CD73/IhrB) and the deletion of the population of cells that was unwanted (Cdl lb). The release markers Cone Arrestin, R/Opsin and Blue opsin expression showed >95% positive for these markers. Furthermore, using the culture conditions described herein, these cells proliferated and allowed for obtaining large number of hCP which were transplanted into the rat eyes. This demonstrated the extremely high capacity of the hCPs to survive and engraft into the host retina. No study prior to the disclosure here, has been able to isolate, culture and transplant human cone progenitor cells and holds great promise for future clinical translation.

Cone progenitor cells were isolated, purified and expanded using a microfluidic based cell sorting approach. Isolating and purifying cone cells is important in the treatment of cone related disorders, as heretofore, there has not been a treatment to replace cones. The enriched, pure population of cone precursors meets the need for providing treatment for various cone related disorders. One example is cone dystrophy which is a degeneration of cone cells, photoreceptor responsible for both central and color vision. Stationary cone dystrophy is usually present during infancy or early childhood and symptoms remain throughout the life. Progressive cone dystrophy is associate when symptoms become worse over time. These symptoms usually develop in late childhood or early adulthood. Age-related macular degeneration, also called macular degeneration,(AMD) is a degenerative disease that causes a progressive loss of central vision. The macula is rich in cones.

Accordingly, the invention provides a unique source of high purity, viable, allogeneic human progenitor cells capable of rescuing and or replacing the functions of damaged or dead cells in many retinal diseases such as cone dystrophy and dry AMD. Key to this disclosure is the isolation, high purity and viability of the cells, manufactured in an efficient and effective system. These cells sustain and or improve vision in patients who are losing or have lost vision. In addition, these cells may be used in drug discovery and screening or other uses where retinal cells or their precursors might be needed. The inventors have been successful with two other cell types, and provides evidence that this approach has broad applicability for the isolation of other cell types throughout the body.

In addition, this is the first time pure viable progenitor cells have been isolated from human embryonic tissues using a specific set of surface markers. A novel two stage technique of sorting allows enriching cone population to greater than 95%. These cells are further cultured using growth factors and other conditions that allow further proliferation.

Accordingly, in one strategy, detailed in the examples section which follows, cone progenitor cells (CPCs) were purified, defined based on a proliferative population expressing cone arrestin and Red/Green (r/g) opsin at greater than 80% using a two multistage approach. CPCs dissociated from embryonic retinas (10-16 weeks) or ES or iPS cell retinal organoids were first sorted based on CD73 expression. After expanding this population, the cells were subjected to a second sort consisting of two different positive antibodies (CD73 and Thrb) and one negative to delete the microglia (CD11b). The positive fraction yielded the new cell line.

The cells and methods embodied herein are not limited to photoreceptor progenitor cells but are applicable to isolation and purification of all progenitor cells.

These cells can be used for cell replacement, drug discovery and screening or other uses where photoreceptors or their precursors might be needed.

This approach had broad applicability for use to isolate other cells in the eye, CNS, and throughout the body. Specifically, the inventors been successful in isolating retinal ganglion cells and retinal microglia, two very difficult targets in the eye.

### Progenitor Cells

Retinal degenerative diseases are generally characterized by the loss of rod and cone photoreceptors, which are the light-detecting cells of the retina (for a review see, Jones M. K. et al., Prog Retin Eye Res. 2017 May; 58:1-27; Tanna P. et al., Br J Ophthalmol. 2017 January; 101(1):25-30). Depending on the disease and genetic mutation involved, rods or cones degenerate first, causing night blindness and tunnel vision or central and daylight vision loss, respectively.

Several approaches such as gene therapy and neuroprotective factors are being explored to prevent or slow down the loss of photoreceptors, but these strategies are generally not restorative, precluding their use in advanced stages of retinal degenerative diseases. In contrast, cell-based therapies offer the potential to restore vision by replacing lost photoreceptors. Studies done in the mammalian retina have highlighted the limitations of endogenous regeneration in higher vertebrates (Ueki Y. et al., Proc Nat/Acad Sci USA. 2015 Nov 3; 112(44):13717-22. Jorstad N. L. et al., Nature. 2017 Aug 3; 548(7665):103 107), but recent studies have taken advantage of the pioneer factor activity of Ascll to trigger Muller glia reprogramming into neurons in the adult retina (Jorstad N. L. et al., 2017). However, the majority of the cells produced by reprogrammed Muller cells in these conditions were not photoreceptors and the overall efficiency of regeneration was low.

Accordingly, in certain embodiments, a method of producing progenitor photoreceptor cells, comprises culturing retinal progenitor cells isolated from a biological sample; isolating CD73⁺cells from the cultured retinal progenitor cells; culturing the CD73+cells; subjecting the CD73⁺cells to a second isolation step comprising isolating CD73⁺+ and CD 11 b⁻cells; culturing and expanding the CD73⁺Thrb⁺CD 11 b⁻cells; to produce the progenitor photoreceptor cells. In certain embodiments, the cells are cone photoreceptor cells.

The protein encoded by the thyroid hormone receptor beta gene (RefSeq NM_000461. HGNC:HGNC:11799. Ensembl:ENSG00000151090 MIM:190160, hereby incorporated by reference) is a nuclear hormone receptor for triiodothyronine. The nucleic acid sequence for human thyroid hormone receptor beta gene can be found at GenBank Accession No.: NC_000001.11 (hereby incorporated by reference). It is one of the several receptors for thyroid hormone, and has been shown to mediate the biological activities of thyroid hormone. Knockout studies in mice suggest that the different receptors, while having certain extent of redundancy, may mediate different functions of thyroid hormone. Mutations in this gene are known to be a cause of generalized thyroid hormone resistance (GTHR), a syndrome characterized by goiter and high levels of circulating thyroid hormone (T3-T4), with normal or slightly elevated thyroid stimulating hormone (TSH). Several alternatively spliced transcript variants encoding the same protein have been observed for this gene.

An amino acid sequence for human Thrb is publically available in the UniProtKB/Swiss-Prot database under accession number P10828.2 (SEQ ID NO: 1) and is as follows:

```
    1 MTPNSMTENG LTAWDKPKHC PDREHDWKLV
        GMSEACLHRK SHSERRSTLK NEQSSPHLIQ
  61 TTWTSSIFHL DHDDVNDQSV SSAQTFQTEE
        KKCKGYIPSY LDKDELCVVC GDKATGYHYR
121 CITCEGCKGF FRRTIQKNLH PSYSCKYEGK
        CVIDKVTRNQ CQECRFKKCI YVGMATDLVL
181 DDSKRLAKRK LIEENREKRR REELQKSIGH
        KPEPTDEEWE LIKTVTEAHV ATNAQGSHWK
241 QKRKFLPEDI GQAPIVNAPE GGKVDLEAFS
        HFTKIITPAI TRVVDFAKKL PMFCELPCED
301 QIILLKGCCM EIMSLRAAVR YDPESETLTL
        NGEMAVTRGQ LKNGGLGVVS DAIFDLGMSL
361 SSFNLDDTEV ALLQAVLLMS SDRPGLACVE
        RIEKYQDSFL LAFEHYINYR KHHVTHFWPK
421 LLMKVTDLRM IGACHASRFL HMKVECPTEL
        FPPLFLEVFE D
```

The CD11 b gene encodes the integrin alpha M chain(HGNC: 6149 Entrez Gene: 3684 Ensembl: ENSG00000169896 OMIM: 120980) (hereby incorporated by reference). Integrins are heterodimeric integral membrane proteins composed of an alpha chain and a beta chain. This I-domain containing alpha integrin combines with the beta 2 chain (ITGB2) to form a leukocyte-specific integrin referred to as macrophage receptor 1 (‘Mac-1’), or inactivated-C3b (iC3b) receptor 3 (′CR3′). The alpha M beta 2 integrin is important in the adherence of neutrophils and monocytes to stimulated endothelium, and also in the phagocytosis of complement coated particles. Multiple transcript variants encoding different isoforms have been found for this gene.

An amino acid sequence for human CD11 b, isoform 1 is publically available in the NCBI GenBank database under accession number NP_00 11 39280.1 (hereby incorporated by reference) (SEQ ID NO: 2) and is as follows:

```
      1 MALRVLLLTA LTLCHGFNLD TENAMTFQEN
          ARGFGQSVVQ LQGSRVVVGA PQEIVAANQR
    61 GSLYQCDYST GSCEPIRLQV PVEAVNMSLG
          LSLAATTSPP QLLACGPTVH QTCSENTYVK
  121 GLCFLFGSNL RQQPQKFPEA LRGCPQEDSD
          IAFLIDGSGS IIPHDFRRMK EFVSTVMEQL
  181 KKSKTLFSLM QYSEEFRIHF TFKEFQNNPN
          PRSLVKPITQ LLGRTHTATG IRKVVRELFN
  241 ITNGARKNAF KILVVITDGE KFGDPLGYED
          VIPEADREGV IRYVIGVGDA FRSEKSRQEL
  301 NTIASKPPRD HVFQVNNFEA LKTIQNQLRE
          KIFAIEGTQT GSSSSFEHEM SQEGFSAAIT
  361 SNGPLLSTVG SYDWAGGVFL YTSKEKSTFI
          NMTRVDSDMN DAYLGYAAAI ILRNRVQSLV
  421 LGAPRYQHIG LVAMFRQNTG MWESNANVKG
          TQIGAYFGAS LCSVDVDSNG STDLVLIGAP
  481 HYYEQTRGGQ VSVCPLPRGQ RARWQCDAVL
          YGEQGQPWGR FGAALTVLGD VNGDKLTDVA
  541 IGAPGEEDNR GAVYLFHGTS GSGISPSHSQ
          RIAGSKLSPR LQYFGQSLSG GQDLTMDGLV
  601 DLTVGAQGHV LLLRSQPVLR VKAIMEFNPR
          EVARNVFECN DQVVKGKEAG EVRVCLHVQK
  661 STRDRLREGQ IQSVVTYDLA LDSGRPHSRA
          VFNETKNSTR RQTQVLGLTQ TCETLKLQLP
  721 NCIEDPVSPI VLRLNFSLVG TPLSAFGNLR
          PVLAEDAQRL FTALFPFEKN CGNDNICQDD
  781 LSITFSFMSL DCLVVGGPRE FNVTVTVRND
          GEDSYRTQVT FFFPLDLSYR KVSTLQNQRS
  841 QRSWRLACES ASSTEVSGAL KSTSCSINHP
          IFPENSEVTF NITFDVDSKA SLGNKLLLKA
  901 NVTSENNMPR TNKTEFQLEL PVKYAVYMVV
          TSHGVSTKYL NFTASENTSR VMQHQYQVSN
  961 LGQRSLPISL VFLVPVRLNQ TVIWDRPQVT
          FSENLSSTCH TKERLPSHSD FLAELRKAPV
1021 VNCSIAVCQR IQCDIPFFGI QEEFNATLKG
          NLSFDWYIKT SHNHLLIVST AEILFNDSVF
1081 TLLPGQGAFV RSQTETKVEP FEVPNPLPLI
          VGSSVGGLLL LALITAALYK LGFFKRQYKD
1141 MMSEGGPPGA EPQ
```

An amino acid sequence for human CD11 b, isoform 2 is publically available in the NCBI GenBank database under accession number NP_000623.2 (hereby incorporated by reference) (SEQ ID NO: 3) and is as follows:

```
      1 MALRVLLLTA LTLCHGFNLD TENAMTFQEN
          ARGFGQSVVQ LQGSRVVVGA PQEIVAANQR
    61 GSLYOCDYST GSCEPIRLQV PVEAVNMSLG
          LSLAATTSPP QLLACGPTVH QTCSENTYVK
  121 GLCFLFGSNL RQQPQKFPEA LRGCPQEDSD
          IAFLIDGSGS IIPHDFRRMK EFVSTVMEQL
  181 KKSKTLFSLM QYSEEFRIHF TFKEFQNNPN
          PRSLVKPITQ LLGRTHTATG IRKVVRELFN
  241 ITNGARKNAF KILVVITDGE KFGDPLGYED
          VIPEADREGV IRYVIGVGDA FRSEKSRQEL
  301 NTIASKPPRD HVFQVNNFEA LKTIQNQLRE
          KIFAIEGTQT GSSSSFEHEM SQEGFSAAIT
  361 SNGPLLSTVG SYDWAGGVFL YTSKEKSTFI
          NMTRVDSDMN DAYLGYAAAI ILRNRVQSLV
  421 LGAPRYQHIG LVAMFRQNTG MWESNANVKG
          TQIGAYFGAS LCSVDVDSNG STDLVLIGAP
  481 HYYEQTRGGQ VSVCPLPRGR ARWQCDAVLY
          GEQGQPWGRF GAALTVLGDV NGDKLTDVAI
  541 GAPGEEDNRG AVYLFHGTSG SGISPSHSQR
          IAGSKLSPRL QYFGQSLSGG QDLTMDGLVD
  601 LTVGAQGHVL LLRSQPVLRV KAIMEFNPRE
          VARNVFECND QVVKGKEAGE VRVCLHVQKS
  661 TRDRLREGQI QSVVTYDLAL DSGRPHSRAV
          FNETKNSTRR QTQVLGLTQT CETLKLQLPN
  721 CIEDPVSPIV LRLNFSLVGT PLSAFGNLRP
          VLAEDAQRLF TALFPFEKNC GNDNICQDDL
  781 SITFSFMSLD CLVVGGPREF NVTVTVRNDG
          EDSYRTQVTF FFPLDLSYRK VSTLQNQRSQ
  841 RSWRLACESA SSTEVSGALK STSCSINHPI
          FPENSEVTFN ITFDVDSKAS LGNKLLLKAN
  901 VTSENNMPRT NKTEFQLELP VKYAVYMVVT
          SHGVSTKYLN FTASENTSRV MQHQYQVSNL
  961 GQRSLPISLV FLVPVRLNQT VIWDRPQVTF
          SENLSSTCHT KERLPSHSDF LAELRKAPVV
1021 NCSIAVCQRI QCDIPFFGIQ EEFNATLKGN
          LSFDWYIKTS HNHLLIVSTA EILFNDSVFT
1081 LLPGQGAFVR SQTETKVEPF EVPNPLPLIV
          GSSVGGLLLL ALITAALYKL GFFKRQYKDM
1141 MSEGGPPGAE PQ
```

Integrin ITGAM/ITGB2 is implicated in various adhesive interactions of monocytes, PGP-23,DNA,M macrophages and granulocytes as well as in mediating the uptake of complement-coated particles and pathogens (PubMed:9558116, PubMed:20008295) (hereby incorporated by reference). It is identical with CR-3, the receptor for the iC3b fragment of the third complement component. It probably recognizes the R-G-D peptide in C3b. Integrin ITGAM/ITGB2 is also a receptor for fibrinogen, factor X and ICAM1. It recognizes P1 and P2 peptides of fibrinogen gamma chain. Regulates neutrophil migration (PubMed:28807980) (hereby incorporated by reference). In association with beta subunit ITGB2/CD18, required for CD177-PRTN3-mediated activation of TNF primed neutrophils (PubMed:21193407) (hereby incorporated by reference). (UniProtKB: P11215 ITAM_HUMAN,hereby incorporated by reference).

CD73 is a cell surface enzyme which is overexpressed in the tumor microenvironment and promotes tumor growth by limiting anti-tumor immunity via the adenosine receptor pathway (HGNC:HGNC:802. Ensembl:ENSG00000135318 MIM:129190. RefSeq NG_028214.1) (hereby incorporated by reference).

A nucleotide sequence that encodes human CD73 is publically available in the NCBI GenBank database under accession number BC065937.1(hereby incorporated by reference) (SEQ ID NO: 4) and is as follows:

```
      1 cgcacccagt tcacgcgcca cagctatgtg
          tccccgagcc gcgcgggcgc ccgcgacgct
    61 actcctcgcc ctgggcgcgg tgctgtggcc
          tgcggctggc gcctgggagc ttacgatttt
  121 gcacaccaac gacgtgcaca gccggctgga
          gcagaccagc gaggactcca gcaagtgcgt
  181 caacgccagc cgctgcatgg gtggcgtggc
          tcggctcttc accaaggttc agcagatccg
  241 ccgcgccgaa cccaacgtgc tgctgctgga
          cgccggcgac cagtaccagg gcactatctg
  301 gttcaccgtg tacaagggcg ccgaggtggc
          gcacttcatg aacgccctgc gctacgatgc
  361 catggcactg ggaaatcatg aatttgataa
          tggtgtggaa ggactgatcg agccactcct
  421 caaagaggcc aaatttccaa ttctgagtgc
          aaacattaaa gcaaaggggc cactagcatc
  481 tcaaatatca ggactttatt tgccatataa
          agttcttcct gttggtgatg aagttgtggg
  541 aatcgttgga tacacttcca aagaaacccc
          ttttctctca aatccaggga caaatttagt
  601 gtttgaagat gaaatcactg cattacaacc
          tgaagtagat aagttaaaaa ctctaaatgt
  661 gaacaaaatt attgcactgg gacattcggg
          ttttgaaatg gataaactca tcgctcagaa
  721 agtgaggggt gtggacgtcg tggtgggagg
          acactccaac acatttcttt acacaggcaa
  781 tccaccttcc aaagaggtgc ctgctgggaa
          gtacccattc atagtcactt ctgatgatgg
  841 gcggaaggtt cctgtagtcc aggcctatgc
          ttttggcaaa tacctaggct atctgaagat
  901 cgagtttgat gaaagaggaa acgtcatctc
          ttcccatgga aatcccattc ttctaaacag
  961 cagcattcct gaagatccaa gcataaaagc
          agacattaac aaatggagga taaaattgga
1021 taattattct acccaggaat tagggaaaac
          aattgtctat ctggatggct cctctcaatc
1081 atgccgcttt agagaatgca acatgggcaa
          cctgatttgt gatgcaatga ttaacaacaa
1141 cctgagacac gcggatgaaa cgttctggaa
          ccacgtatcc atgtgcattt taaatggagg
1201 tggtatccgg tcgcccattg atgaacgcaa
          caatggcaca attacctggg agaacctggc
1261 tgctgtattg ccctttggag gcacatttga
          cctagtccag ttaaaaggtt ccaccctgaa
1321 gaaggccttt gagcatagcg tgcaccgcta
          cggccagtcc actggagagt tcctgcaggt
1381 gggcggaatc catgtggtgt atgatctttc
          ccgaaaacct ggagacagag tagtcaaatt
1441 agatgttctt tgcaccaagt gtcgagtgcc
          cagttatgac cctctcaaaa tggacgaggt
1501 atataaggtg atcctcccaa acttcctggc
          caatggtgga gatgggttcc agatgataaa
1561 agatgaatta ttaagacatg actctggtga
          ccaagatatc aacgtggttt ctacatatat
1621 ctccaaaatg aaagtaattt atccagcagt
          tgaaggtcgg atcaagtttt ccacaggaag
1681 tcactgccat ggaagctttt ctttaatatt
          tctttcactt tgggcagtga tctttgtttt
1741 ataccaatag ccaaaaattc tccttgcctt
          taatgtgtga aactgcattt tttcaagtga
1801 gattcaaatc tgccttttag gacctggctt
          tgtgacagca aaaaccatct ttacaggctc
1861 ctagaagctg aaggttagag cattataaaa
          tgaagagaca gacatgatta ctcagggtca
1921 gcaacctagt gagttagaaa aaaaattaac
          atagggccct ataaggagaa agccaactat
1981 gttaagttta tgtgtccaaa ttttaatgaa
          attttactaa caattttaaa ccatattttt
2041 cttcttcata tccatttcta atccatcaaa
          cagcttatgt ttacataaaa ttttatcatt
2101 cacaaggaag ttttaagcac actgtctcat
          ttgatatcca caacttattt ttggtaggaa
2161 agagagatgt ttttcccacc tgtcagatga
          aaaaactgaa gctcaaaaag ggttgacttg
2221 accatacagc taatgctgac agatccaaga
          cctagaccta ggtcttttga actcaagtcc
2281 aggattctca actatatcaa gttactgttc
          agaatactta atatctcctc tcttcataat
2341 tatcaatagc cccaagctca tggatgacaa
          atctctgctt tatttcttgt ctctattttt
2401 tcactttata gctcctgtta taatagcaag
          tttaatggta taaacacagg ataccatcct
2461 ctcttgcaac acccatgtgc ctttgatgag
          tcaggtagca agctgtagta gataatgaga
2521 aaggccagag gctgcaaaag acagtcaaag
          gacacgagag aaaggaaggg gaagaacagg
2581 actccaggac tgttttatat tatagaaaag
          caagagctaa agagcattta cacatgttaa
2641 acagatactt gttaagcata gtgcctgaca
          cacggcatta gctgttatga gattccatca
2701 gctctgcctc tgtcctcttt cttctaacat
          gaaggtatca tgagaagaga accttctaac
2761 ataagctgta attctaaacc tgcacttgtc
          cctctccagc aagaggctag cactgaattc
2821 attctactca tactacacac ccagttatgg
          aatgtccaga gttctcgaag aaaataaatg
2881 actttaggaa gaggtataca ttttttaagt
          cgctctgcct ccaaatctga acagtcactg
2941 taaatcattc ttaagcccag atatgagaac
          ttctgctgga aagtgggacc ctctgagtgg
3001 gtggtcagaa aatacccatg ctgatgaaat
          gacctatgcc caaagaacaa atacttaacg
3061 tgggagtgga accacatgag cctgctcagc
          tctgcataag taattcaaga aatgggaggc
3121 ttcaccttaa aaacagtgtg caaatggcag
          ctagaggttt tgataggaag tatgtttgtt
3181 tcttagtgtt tacaaatatt aagtactctt
          gatacaaaat atacttttaa acttcataac
3241 ctttttataa aagttgttgc agcaaaataa
          tagcctcggt tctatgcata tatggattag
3301 ctataaaaaa tgtcaataag attgtacaag
          gaaaattaga gaaaggcaca tttagggttt
3361 attttttaca cttggccagt aaaatagggt
          aaatcctatt agaatttttt aaagaacttt
3421 ttttaagttt cctaaatctg tgtgtgtatt
          gtgaagtggt ataagaaatg actttgaacc
3481 actttgcaat tgtagattcc caacaataaa
          attgaagata aaaaaaaaaa aaaaaaaaaa
3541 aaaaaaaaaa a
```

An amino acid sequence for human CD73 is publically available in the NCBI GenBank database under accession number AAH65937.1 (hereby incorporated by reference) (SEQ ID NO: 5) and is as follows (the signal peptide is amino acids 1-26 which are bold and underlined):

```
    1 MCPRAARAPA TLLLALGAVL WPAAGAWELT
        ILHTNDVHSR LEQTSEDSSK CVNASRCMGG
  61 VARLFTKVQQ IRRAEPNVLL LDAGDQYQGT
        IWFTVYKGAE VAHFMNALRY DAMALGNHEF
121 DNGVEGLIEP LLKEAKFPIL SANIKAKGPL
        ASQISGLYLP YKVLPVGDEV VGIVGYTSKE
181 TPFLSNPGTN LVFEDEITAL QPEVDKLKTL
        NVNKIIALGH SGFEMDKLIA QKVRGVDVVV
241 GGHSNTFLYT GNPPSKEVPA GKYPFIVTSD
        DGRKVPVVQA YAFGKYLGYL KIEFDERGNV
301 ISSHGNPILL NSSIPEDPSI KADINKWRIK
        LDNYSTQELG KTIVYLDGSS QSCRFRECNM
361 GNLICDAMIN NNLRHADETF WNHVSMCILN
        GGGIRSPIDE RNNGTITWEN LAAVLPFGGT
421 FDLVQLKGST LKKAFEHSVH RYGQSTGEFL
        QVGGIHVVYD LSRKPGDRVV KLDVLCTKCR
481 VPSYDPLKMD EVYKVILPNF LANGGDGFQM
        IKDELLRHDS GDQDINVVST YISKMKVIYP
541 AVEGRIKFST GSHCHGSFSL IFLSLWAVIF
        VLYQ
```

Other markers for cone photoreceptor cells, include without limitation: RXRG PGP-25,DNA (retinoid X receptor (RXR) gamma), THRB (thyroid hormone receptor beta), SALL3 (Sall-like protein 3), ONECUT1 (One cut domain, family member 1), OPN1SW (Opsin 1 short wavelength), OPN 1 LW/MW (Opsin 1 long wavelength and medium wavelength), ARR3 (Arrestin -C-3), GNAT2 (G—protein subunit alpha transducing 1), CNGB3 (cyclic nucleotide-gated (CNG) channel beta subunit), PDE6H (phosphodiesterase 6H), PDE6C (phosphodiesterase 6C), GUCA1A (guanylate cyclase activator 1A).

An amino acid sequence for human Cone Arrestin is publically available in the UniProtKB/Swiss-Prot database under accession number P36575.2(hereby incorporated by reference) (SEQ ID NO: 6) and is as follows:

```
    1 MSKVFKKTSS NGKLSIYLGK RDFVDHVDTV
        EPIDGVVLVD PEYLKCRKLF VMLTCAFRYG
  61 RDDLEVIGLT FRKDLYVQTL QVVPAESSSP
        QGPLTVLQER LLHKLGDNAY PFTLQMVTNL
121 PCSVTLQPGP EDAGKPCGID FEVKSFCAEN
        PEETVSKRDY VRLVVRKVQF APPEAGPGPS
181 AQTIRRFLLS AQPLQLQAWM DREVHYHGEP
        ISVNVSINNC TNKVIKKIKI SVDQITDVVL
241 YSLDKYTKTV FIQEFTETVA ANSSFSQSFA
        VTPILAASCQ KRGLALDGKL KHEDTNLASS
301 TIIRPGMDKE LLGILVSYKV RVNLMVSCGG
        ILGDLTASDV GVELPLVLIH PKPSHEAASS
361 EDIVIEEFTR KGEEESQKAV EAEGDEGS
```

An amino acid sequence for human medium wave (green) Opsin is publically PGP-26,DNA available in the NCBI GenBank database under accession number NP_001041646.1(hereby incorporated by reference) (SEQ ID NO: 7) and is as follows:

```
    1 MAQQWSLQRL AGRHPQDSYE DSTQSSIFTY
        TNSNSTRGPF EGPNYHIAPR WVYHLTSVWM
  61 IFVVIASVFT NGLVLAATMK FKKLRHPLNW
        ILVNLAVADL AETVIASTIS VVNQVYGYFV
121 LGHPMCVLEG YTVSLCGITG LWSLAIISWE
        RWMVVCKPFG NVRFDAKLAI VGIAFSWIWA
181 AVWTAPPIFG WSRYWPHGLK TSCGPDVFSG
        SSYPGVQSYM IVLMVTCCIT PLSIIVLCYL
241 QVWLAIRAVA KQQKESESTQ KAEKEVTRMV
        VVMVLAFCFC WGPYAFFACF AAANPGYPFH
301 PLMAALPAFF AKSATIYNPV IYVFMNRQFR
        NCILQLFGKK VDDGSELSSA SKTEVSSVSS
361 VSPA
```

An amino acid sequence for human long wave (red) Opsin is publically available in PGP-27,DNA the NCBI GenBank database under accession number NP_064445.2 (hereby incorporated by reference) (SEQ ID NO: 8) and is as follows:

```
    1 MAQQWSLQRL AGRHPQDSYE DSTQSSIFTY
        TNSNSTRGPF EGPNYHIAPR WVYHLTSVWM
  61 IFVVTASVFT NGLVLAATMK FKKLRHPLNW
        ILVNLAVADL AETVIASTIS IVNQVSGYFV
121 LGHPMCVLEG YTVSLCGITG LWSLAIISWE
        RWMVVCKPFG NVRFDAKLAI VGIAFSWIWA
181 AVWTAPPIFG WSRYWPHGLK TSCGPDVFSG
        SSYPGVQSYM IVLMVTCCII PLAIIMLCYL
241 QVWLAIRAVA KQQKESESTQ KAEKEVTRMV
        VVMIFAYCVC WGPYTFFACF AAANPGYAFH
301 PLMAALPAYF AKSATIYNPV IYVFMNRQFR
        NCILQLFGKK VDDGSELSSA SKTEVSSVSS
361 VSPA
```

An amino acid sequence for human rhodopsin is publically available in the NCBI GenBank database under accession number NP_000530.1 (hereby incorporated by reference) (SEQ ID NO: 9) and is as follows:

```
    1 MNGTEGPNFY VPFSNATGVV RSPFEYPQYY
        LAEPWQFSML AAYMFLLIVL GFPINFLTLY
  61 VTVQHKKLRT PLNYILLNLA VADLFMVLGG
        FTSTLYTSLH GYFVFGPTGC NLEGFFATLG
121 GEIALWSLVV LAIERYVVVC KPMSNFRFGE
        NHAIMGVAFT WVMALACAAP PLAGWSRYIP
181 EGLQCSCGID YYTLKPEVNN ESFVIYMFVV
        HFTIPMIIIF FCYGQLVFTV KEAAAQQQES
241 ATTQKAEKEV TRMVIIMVIA FLICWVPYAS
        VAFYIFTHQG SNFGPIFMTI PAFFAKSAAI
301 YNPVIYIMMN KQFRNCMLTT ICCGKNPLGD
        DEASATVSKT ETSQVAPA
```

Accordingly, in these and other embodiments, a cone photoreceptor cell comprises one or more markers comprising: RXRG, THRB, SALL3, ONECUT1, OPNISW, OPN 1 LW/MW, ARR3, GNAT2, CNGB3, PDE6H, PDE6C, GUCA1A or combinations thereof. In certain embodiments, an early cone photoreceptor cell comprises one or more markers comprising: RXRG, THRB, SALL3, ONECUT1 or combinations thereof. The markers: RXRG, THRB, SALL3, ONECUT1 are markers for early cone photoreceptor. The markers: OPN 1 SW, OPN 1 LW/MW, ARR3, GNAT2, CNGB3, PDE6H, PDE6C, GUCA1A are markers for mature cone photoreceptor cells.

It is to be understood that progenitor cells from any biological sample can be isolated and purified by the methods embodied herein. Accordingly, in certain embodiments, a method of isolating and purifying viable progenitor cells, comprises isolating cells from a biological sample; culturing and expanding the cells; isolating cells based on a first biomarker profile and further culturing of the isolated cells; subjecting the cultured isolated cells to a second isolation step based on a second biomarker profile; thereby, producing a purified population of progenitor cells. In these and other embodiments, the biological sample comprises: fetal tissues, embryonic tissues, extraembryonic, tissues, cord blood, fluids, cord tissues, bone marrow, adult tissues or combinations thereof.

Examples of markers useful to practice embodiments of the invention are as follows:

Embryonic Stem Cells: Embryonic stem cells (ES cells) are pluripotent stem cells derived from the inner cell mass of a blastocyst, an early-stage embryo. The ES cells have high potential to differentiate into a wide variety of cell types. Undifferentiated embryonic stem (ES) and induced pluripotent stem (iPS) cells can be functionally defined by their ability to differentiate into cells derived from the three germ layers the ectoderm, mesoderm, and endoderm that eventually make up to all cell types at a later time. ES cells possess two distinct properties that make them an attractive choice for cell therapy. First, as embryonic stem cells originate from early blastocysts, a very early developmental stage, retain the extraordinary plasticity to become any cell type of approximately 200 cell types that constitute the human body. Given the right combination of signals, embryonic stem cells will develop into mature cells that can function as neurons, muscles, bone, blood or other needed cell types. Another important feature of embryonic stem cells is their ability to remain in an undifferentiated state and to divide indefinitely. These so-called self-renewing cells generate unlimited well-defined, identical, genetically and genomically characterized stem cells.

Cell surface pluripotency markers. Two homeodomain transcription factors, Oct4 and Nanog, were the first proteins identified as essential for both early embryo development and pluripotency maintenance in ES cells. In addition to Oct4, Sox2, and Nanog, many other factors required for pluripotency have been identified, including Sa114, Daxl, Essrb, Tbx3, Tcll, Rifl, Nacl, and Zfp281.

TRA-1-60 and TRA-1-81. TRA-1-60 and TRA-1-81 antigens on the human embryonal carcinoma (EC) cells and human pluripotent stem cell surfaces can be used as markers in identifying and isolating ESCs. They are also routinely used to assess the pluripotency status of induced pluripotent stem (iPS) cells. Both TRA-1-60 and SSEA4 are both expressed on human embryonal carcinomas and on human embryonic stem cells. Upon differentiation, TRA-1-60 and SSEA4 expression levels decrease and SSEA1 expression increases on human embryonic stem cells over time when treated with Retinoic acid. Besides, they also express CD349/frizzled-9, stage-specific embryonic antigen (SSEA)-4, Oct-4, Nanog, and nestin. Oct-4 and Nanog, as well as several cell surface markers (SSEA-1, SSEA-4, TRA-1-60, and TRA1-81) have been used to characterize mouse and human embryonic stem cells (ESC).

Stage-specific embryonic antigens (SSEA). Conventionally, markers used for mESCs, mouse embryonic carcinomas (ECs), or human EC cells, were exploited to identify undifferentiated human embryonic stem cells (hESCs). At the pre-implantation stage, murine embryos, human germ cells, and teratocarcinoma stem cells express certain molecular receptors known as Stage-Specific Embryonic Antigens (SSEA) on their membrane surface. It is now widely recognized that SSEAs, sphingolipids, identified as a key player in identifying cells endowed with pluripotent and stem cell characteristics. During oogenesis and embryogenesis, SSEA-3 and SSEA-4 are expressed in undifferentiated primate ESC, human embryonic germ (EG) cells, human teratocarcinoma stem cells, and ESC. Currently, Stage-specific embryonic antigen-3 (SSEA-3) and SSEA-4 have been recognized to characterize undifferentiated hESCs but not on undifferentiated mESCs.

Frizzled (Fzd) and Cripto (7DGF-1). A family of Frizzled proteins is expressed in mouse and human ESC. Wnt signals execute their functions via the Fzd family receptors by bind to Fzd and the co-receptors LRP5 or LPR6, and eventually activate the Wnt/β-catenin pathway. Cripto-1 plays a crucial role for early embryonic development and has been associated with the undifferentiated status of mouse ES and human ES cells. During development, Cripto acts as a receptor for TGF-β ligands, including GDF1 and GDF3. In addition to having essential functions during embryogenesis, as an oncogene, Cripto is upregulated in tumors and promotes tumorigenesis. Frizzled (FZD) proteins comprise a family of transmembrane-spanning receptors (FZD1-10) activated by Wnt ligands. FZD9 can be activated by Wnt 2 and Wnt 8 via the canonical pathway, and by Wnt 7 via non-canonical signaling. Mouse FZD9 is present in the developing brain, in neural precursor cells in the developing neural tube, and in myotomes.

Transcription factors. The fundamental implication of transcription factors in the pluripotency maintenance was clearly exemplified by the fact that pluripotent stem cells can be derived from mouse embryonic fibroblasts by inducing transcription factors expression. In addition to extrinsic factors, the pluripotency of ES cells also depends on intrinsic determinants, such as the expression of the POU transcription factor. As such, these induced pluripotent stem (iPS) cells were developed via the overexpression of a set of specific genes Oct4, c-Myc, Sox2, and Klf4. Abundant levels of Oct4, Sox2, and Klf4 reprogram fibroblasts into the iPS cells with a pluripotent state.

A combination of either Klf4 or c-Myc with Oct4 is sufficient to generate iPS cells from NSC. Oct3/4 is specifically expressed in pluripotent stem cells. The Sox family of genes is associated with multipotent and unipotent stem cells. Sox1 induces iPS cells with similar efficiency as Sox2, and genes Sox3, Sox 15, and Sox 18 generate iPS cells, although with decreased efficiency. Klf5 has been implicated in the transcription of Oct3/4 and Nanog, and ESCs renewal and pluripotency maintenance. Klf4 and Klf2 can regulate the expression of certain transcription factors: Nanog, Tcll, Esrrb, Sa114, Tcf3, Mycn and Fbxo15. Nanog is a transcription factor and plays a crucial role in the maintenance of pluripotency and self-renewal in mouse and human ESCs and is downregulated upon ESC differentiation, which is consistent with an intimate association with pluripotent stem cell identity. Nanog has been implicated in pluripotent ES and EG cells, as well as in both mouse and human EC cells.

Induced-PS cell markers. Fully reprogrammed human iPS cells, compared to transiting or incompletely reprogrammed cells, are endowed with the important features (i) downregulation of CD 13, a fibroblast marker, (ii) upregulation of expression of SSEA-4 and TRA-1-60like pluripotent markers, (iii) silencing of viral transgenes (iv) endogenous expression of Nanog (v) a low Hoechst retaining or high Hoechst pump out potential. Similar to expression pattern found in hESC, human iPSCs also express the SSEA-3, SSEA-4, TRA-1-60, TRA-1-81, TRA-2-49/6E, and Nanog. Gene expression and genome-wide H3K4me3 and H3K27me3 were found to be extremely similar between ES and iPS cells/. In mouse, iPSCs expressed genes expressed in undifferentiated ESCs, including Oct-3/4, Sox2, Nanog, GDF3, REX1, FGF4, ESG1, DPPA2, DPPA4, and hTERT. Similar to mESCs, mouse iPSCs do not express SSEA-3 and SSEA-4 but both cell types commonly express SSEA-1.

Germ cell markers. The precursors of primordial germ cells developed from 4-8 cells in E6.25 proximal epiblast express the transcriptional repressor Blimpl (Ohinata Y. et al., Nature. 2005; 436:207-13). Over time, these Blimpl-positive cells continuously proliferate and initiate the expression of Fragilis and Stella by E7.5 (Mise N et al., Genes Cells. 2008; 13:863-77). Reports indicate that the expression of Blimpl, Stella, Fragilis, Piwil2, Dazl, and MVH in ES cells (Mise N et al., 2008; Geijsen N. et al., Nature. 2004; 427:148-54), indicating the origin of ES cells could be germline. Depending on the stage, certain markers like Oct4, c-kit are expressed early and downregulated prior to mature Germline Specification stages. Tekt 1 and GDF9 markers are induced only at later stages. Nanos has been implicated in all development stages from blastocyst to mature sperm or oocyte.

Ectoderm and endoderm markers. Ectoderm is one of the three primary germ cell layers in the very early embryo. The other two layers are the mesoderm (middle layer) and endoderm (most proximal layer), with the ectoderm as the most exterior (or distal) layer. Certain factors mark the ectoderm including Otx2, Chordin, p63/TP73L, FGF-8, Pax2. FoxJ3, Pax6, GBX2, SOX1, Nestin, beta- Tubulin, and Noggin. Endoderm formation depends on two sequential positive feedback loops mediated by Cripto and Bmp4/Wnt3 that are activated by mature or uncleaved Nodal, respectively, to sustain Nodal signaling from implantation throughout gastrulation (Ben Haim N et al., Dev Cell. 2006; 11:313-23). ENDM1 and Flkl have been used as a definitive mouse endodermal cell marker and a mesoderm cell marker, respectively (Nicetto D. et al., Science. 2019; 363:294-297).

Enzymatic and other relevant marker systems. Several additional marker systems useful in the isolation and purification of progenitor cells by the methods embodied herein, include enzymatic (alkaline phosphatase and telomerase)-based reaction, small molecules (lectins or short peptides), and quantum dots (QD) or fluorescence dyes, etc.

Hematopoietic Stem Cell Markers. Hematopoietic stern cells (HSCs) are unique, multipotent, self-renewing progenitor cells responsible for continuous supply of differentiated blood cell types of the myeloid and lymphoid lineages. These cells include lymphocytes, granulocytes, and macrophages of the immune system as well as circulating erythrocytes and platelets. HSCs are defined by two key functional abilities: (i) multipotency - the ability to form all differentiated blood cells, and (ii) long-term self-renewal —the ability to give rise to identical daughter cell that of the ancestor. I—ISC is superior with self-renewal that distinguishes HSC from multipotent progenitors and is responsible for successful secondary transplants in experimental models. A major challenge using Hematopoietic Stem Cells (HSCs) is their identification and isolation from larger pools of cells as HSCs represent a very rare, 1 in 10,000 cells of the hone marrow and 1 in 100,000 cells in the blood.

Therefore the methods embodied herein are useful in isolating and purifying large scale viable HSCs.

Examples of biomarkers that can be targeted for the isolation and enrichment of HSCs include: SLAM (Signaling Lymphocyte Activation Molecule) family of cell surface molecules which includes CD48, CD150, CD244, etc.

Lymphoid lineage markers. Hematopoietic Stem Cells (HSCs) can differentiate into cells of two primary lineages, lymphoid and myeloid. Common lymphoid progenitors can differentiate into all lymphoid lineages (Kondo M. et al., Cell. 1997; 91:661-72). During the process of lymphopoiesis, lymphoid lineage leads to the formation of B Cells, T Cells, Natural Killer (NK) Cells, and Dendritic Cells. Many of these lymphocytes are short-lived, and immune system homeostasis requires continual HSC self-renewal and differentiation. The markers, c-Kitt^{Lo}, Sca-1^{Lo}, Lin and IL7R⁺ have shown to represent lymphoid lineage cells.

Myeloid lineage markers. Common myeloid progenitors differentiate into progenitors then can differentiate into the granulocyte/macrophage and megakaryocyte/erythroid lineages, respectively (Akashi K. et al., Nature. 2(x)( );404:193-7). During the process of myelopoiesis, the myeloid lineage develops to form Granulocytes, Monocytes, Megakaryocytes, and Dendritic cells. Circulating erythivcytes and platelets also develop from myeloid progenitor cells. Many of these myeloid cells are short-lived, and immune system homeostasis requires continual HSC self-renewal and differentiation.

### Candidate Agents and Screening Assays

The compositions embodied herein, can also be applied in the areas of drug discovery and target validation. The present disclosure comprehends the use of the progenitor cells, nucleic acid sequences and peptides, in drug discovery efforts to elucidate relationships that exist in a disease state, e.g. macular degeneration, and the regeneration or differentiation of a progenitor cell.

The screening assays of the disclosure suitably include and embody, animal models, cell-based systems and non-cell based systems. The progenitor cells embodied herein, are used for identifying agents of therapeutic interest, e.g. by screening libraries of compounds or otherwise identifying compounds of interest by any of a variety of drug screening or analysis techniques, or synthesis of novel compounds. (Moffat, J. G., et al., (2014). Phenotypic screening in cancer drug discovery —past, present and future. Nature Reviews Drug Discovery, 13(8), 588-602. doi: 10.1038/nrd4366; Swinney, D. C., & Anthony, J. (2011). How were new medicines discovered—Nature Reviews Drug Discovery, 10(7), 507-519. doi: 10.1038/nrd3480; Chackalamannil, S., et al., (2017). Comprehensive medicinal chemistry III. Amsterdam: Elsevier, E. L. Berg, et al., “Approaches to the analysis of cell signaling networks and their application in drug discovery.,” Current Opinion in Drug Discovery & Development, vol. 8, no. 1, pp. 107-114, 2005)

The assays can be of an in vitro or in vivo format. In vitro formats of interest include cell-based formats, in which contact occurs e.g., by introducing the substrate in a medium, such as an aqueous medium, in which the cell is present. In yet other embodiments, the assay may be in vivo, in which a multicellular organism that includes the cell is employed.

Multicellular organisms of interest include, but are not limited to: insects, vertebrates, such as avian species, e.g., chickens; mammals, including rodents, e.g., mice, rates; ungulates, e.g., pigs, cows, horses; dogs, cats, primates, e.g., monkeys, apes, humans; and the like. As such, the target cells of interest include, but are not limited to: insects cells, vertebrate cells, particularly avian cells, e.g., chicken cells; mammalian cells, including murine, porcine, ungulate, ovine, equine, rat, dog, cat, monkey, and human cells; and the like.

In certain embodiments, the subject methods are performed in a high throughput (HT) format. In the subject HT embodiments of the subject invention, a plurality of different cells are simultaneously assayed or tested. By simultaneously tested is meant that each of the cells in the plurality are tested at substantially the same time. In general, the number of cells that are tested simultaneously in the subject HT methods ranges from about 10 to 10,000, usually from about 100 to 10,000 and in certain embodiments from about 1000 to 5000. A variety of high throughput screening assays for determining the activity of candidate agent are known in the art and are readily adapted to the present invention.

In certain embodiments, a detectable moiety is conjugated to an agent of interest wherein the detectable moiety comprises: a luminescent moiety, a chemiluminescent moiety, a fluorescence moiety, a bioluminescent moiety, an enzyme, a natural or synthetic moiety.

Candidate Agents: The methods can be practiced with any test compounds as candidate agents. Test compounds useful in practicing the inventive method may be obtained using any of the numerous approaches in combinatorial library methods known in the art, including biological libraries, spatially-addressable parallel solid phase or solution phase libraries, synthetic library methods requiring deconvolution, the “one-bead one-compound” library method, and synthetic library methods using affinity chromatography selection. The biological library approach is limited to peptide libraries, while the other four approaches are applicable to peptide, nonpeptide oligomer, or small molecule libraries of compounds (Lam, 1997, Anticancer Drug Des. 12:145).

Examples of methods for the synthesis of molecular libraries may be found in the art, for example, in: DeWitt et al., 1993, Proc. Natl. Acad. Sci. USA 90:6909-6913; Erb et al., 1994, Proc. Natl. Acad. Sci. USA 91:11422-11426; Zuckermann et al., 1994, J. Med. Chem. 37:2678-2685; Cho et al., 1992, Science 261:1303-1305; Carell et al., 1994, Angew. Chem. hit. Ed. Engl. 33:2059-2061; Carell et al., 1994, Angew. Chem. Int. Ed. Engl. 33:2061-2064; and Gallop et al., 1994, J. Med. Chem. 37:1233-1251. Libraries of compounds may be presented in solution (e.g., Houghten, 1992, Bio/Techniques 13:412-421), or on beads (Lam, 1991, Nature 354:82-84), chips (Fodor, 1993, Nature 364:555-556), bacteria (U.S. Pat. No.¶5,223,409), spores (U.S. Pat. Nos. 5,571,698; 5,403,484; and 5,223,409), plasmids (Cull et al., 1992, Proc. Natl. Acad. Sci. USA 89:1865-1869), or phage (Scott and Smith, 1990, Science 249:386-390; Devlin, 1990, Science 249:404-406; Cwirla et al., 1990, Proc. Natl. Acad. Sci. USA 87:6378-6382; and Felici, 1991, J Mol. Biol. 222:301-310).

Commercially available libraries that may be screened include, but are not limited to, the TimTec Natural Product Library (NPL), NPL-640, and TimTec NDL-3000 library. Libraries comprising compounds modeled on polyamines (i.e., polyamine analogs) may also be screened.

In certain embodiments, the candidate agent is a small molecule or large molecule ligand. By small molecule ligand is meant a ligand ranging in size from about 50 to about 10,000 daltons, usually from about 50 to about 5,000 daltons and more usually from about 100 to about 1000 daltons. By large molecule is meant a ligand ranging in size from about 10,000 daltons or greater in molecular weight.

The method may be practiced iteratively using different concentrations of a test candidate and/or different testing conditions, such as duration of reaction time. Test candidates that are identified by the method can be further tested by conventional methods in the art to verify specificity, dose dependency, efficacy in vivo, and the like. Test candidates may serve as lead compounds for developing additional test candidates.

A prototype compound or agent may be believed to have therapeutic activity on the basis of any information available to the artisan. For example, a prototype agent may be believed to have therapeutic activity on the basis of information contained in the Physician's Desk Reference. In addition, by way of non-limiting example, a compound may be believed to have therapeutic activity on the basis of experience of a clinician, structure of the compound, structural activity relationship data, EC50, assay data, IC50 assay data, animal or clinical studies, or any other basis, or combination of such bases.

A therapeutically-active compound or agent is an agent that has therapeutic activity, including for example, the ability of the agent to induce a specified response when administered to a subject or tested in vitro. Therapeutic activity includes treatment of a disease or condition, including both prophylactic and ameliorative treatment. Treatment of a disease or condition can include improvement of a disease or condition by any amount, including prevention, amelioration, and elimination of the disease or condition. Therapeutic activity may be conducted against any disease or condition, including in a preferred embodiment against any disease or disorder associated with damage by reactive oxygen intermediates. In order to determine therapeutic activity any method by which therapeutic activity of a compound may be evaluated can be used. For example, both in vivo and in vitro methods can be used, including for example, clinical evaluation, EC5O, and IC50 assays, and dose response curves.

Candidate compounds for use with an assay of the present disclosure or identified by assays of the present disclosure as useful pharmacological agents can be pharmacological agents already known in the art or variations thereof or can be compounds previously unknown to have any pharmacological activity. The candidate compounds can be naturally occurring or designed in the laboratory. Candidate compounds can comprise a single diastereomer, more than one diastereomer, or a single enantiomer, or more than one enantiomer.

Candidate compounds can be isolated, from microorganisms, animals or plants, for example, and can be produced recombinantly, or synthesized by chemical methods known in the art. If desired, candidate compounds of the present disclosure can be obtained using any of the numerous combinatorial library methods known in the art, including but not limited to, biological libraries, spatially addressable parallel solid phase or solution phase libraries, synthetic library methods requiring deconvolution, the “one-bead one-compound” library method, and synthetic library methods using affinity chromatography selection. The biological library approach is limited to polypeptide libraries. The other four approaches are applicable to polypeptide, non-peptide oligomers, or small molecule libraries of compounds and are preferred approaches in the present disclosure. See Lam, Anticancer Drug Des. 12: 145-167 (1997).

In an embodiment, the present disclosure provides a method of identifying a candidate compound as a suitable prodrug. A suitable prodrug includes any prodrug that may be identified by the methods of the present disclosure. Any method apparent to the artisan may be used to identify a candidate compound as a suitable prodrug.

In another aspect, the present disclosure provides methods of screening candidate compounds for suitability as therapeutic agents. Screening for suitability of therapeutic agents may include assessment of one, some or many criteria relating to the compound that may affect the ability of the compound as a therapeutic agent. Factors such as, for example, efficacy, safety, efficiency, retention, localization, tissue selectivity, degradation, or intracellular persistence may be considered. In an embodiment, a method of screening candidate compounds for suitability as therapeutic agents is provided, where the method comprises providing a candidate compound identified as a suitable prodrug, determining the therapeutic activity of the candidate compound, and determining the intracellular persistence of the candidate compound. Intracellular persistence can be measured by any technique apparent to the skilled artisan, such as for example by radioactive tracer, heavy isotope labeling, or LCMS.

In screening compounds for suitability as therapeutic agents, intracellular persistence of the candidate compound is evaluated. In an embodiment, the agents are evaluated for their ability to modulate the translation of compositions embodied herein, over a period of time in response to a candidate therapeutic agent. In other embodiments, the candidate therapeutic agent is neuroprotective, increases survivability of the cells, induces engraftment of cells etc.

In another preferred embodiment, soluble and/or membrane-bound forms of compositions embodied herein, e.g. proteins, mutants or biologically active portions thereof, can be used in the assays for screening candidate agents. When membrane-bound forms of the protein are used, it may be desirable to utilize a solubilizing agent. Examples of such solubilizing agents include non-ionic detergents such as n-octylglucoside, n-dodecylglucoside, n-dodecylmaltoside, octanoyl-N-methylglucamide, decanoyl-N-methylglucamide, TRITON™ X-100, TRITON™ X-114, THESIT, Isotridecypoly(ethylene glycol ether)n, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl=N,N-dimethyl-3-ammonio-l-propane sulfonate.

### Disorders

In certain embodiments, a method of treating an ocular or retinal disease comprises administering to a subject an effective amount of the isolated and purified population of progenitor cells embodied herein.

Ocular disorders that can be treated using a method of the present disclosure include, but are not limited to, macular degeneration, choroidal neovascularization, macular edema, retinal neovascularization, proliferative vitreoretinopathy, glaucoma, and ocular inflammation.

Ocular diseases that can be treated using a method of the present disclosure include, but are not limited to, acute macular neuroretinopathy; Behcet's disease; choroidal neovascularization; diabetic uveitis; histoplasmosis; macular degeneration, such as acute macular degeneration, non-exudative age related macular degeneration and exudative age related macular degeneration; edema, such as macular edema, cystoid macular edema and diabetic macular edema; multifocal choroiditis; ocular trauma which affects a posterior ocular site or location; ocular tumors; retinal disorders, such as central retinal vein occlusion, diabetic retinopathy (including proliferative diabetic retinopathy and diabetic macular edema), proliferative vitreoretinopathy (PVR), retinal arterial occlusive disease, retinal detachment, uveitic retinal disease; sympathetic ophthalmia; Vogt Koyanagi-Harada (VKH) syndrome; uveal diffusion; a posterior ocular condition caused by or influenced by an ocular laser treatment; posterior ocular conditions caused by or influenced by a photodynamic therapy; photocoagulation, radiation retinopathy; epiretinal membrane disorders; branch retinal vein occlusion; anterior ischemic optic neuropathy; non-retinopathy diabetic retinal dysfunction; retinoschisis; retinitis pigmentosa; glaucoma; Usher syndrome, cone-rod dystrophy; Stargardt disease (fundus flavimaculatus); inherited macular degeneration; chorioretinal degeneration; Leber congenital amaurosis; congenital stationary night blindness; choroideremia; Bardet-Biedl syndrome; macular telangiectasia; Leber's hereditary optic neuropathy; retinopathy of prematurity; and disorders of color vision, including achromatopsia, protanopia, deuteranopia, and tritanopia.

In some cases, the ocular disease is glaucoma, retinitis pigmentosa, macular degeneration, retinoschisis, Leber's Congenital Amaurosis, diabetic retinopathy, achromotopsia, or color blindness.

Embodiments of the invention are also directed to treatment of any disease or disorder wherein VEGF is protective such as neurodegenerative diseases, e.g. Niemann-Pick disease, Alzheimer's disease, Parkinson's disease, Lou Gehrig's disease, schizophrenia, Gaucher disease, Fabry disease, Tay-Sachs disease, Sandhoff disease and cerebellar ataxia, but is not limited thereto.

### Administration of Compositions

The compositions of the present disclosure can be prepared in a variety of ways known to one of ordinary skill in the art. Regardless of their original source or the manner in which they are obtained, the compositions of the disclosure can be formulated in accordance with their use. For example, the progenitor cells, therapeutic agents etc., above can be formulated within compositions for application to cells in tissue culture or for administration to a patient or subject. Any of the pharmaceutical compositions of the disclosure can be formulated for use in the preparation of a medicament, and particular uses are indicated below in the context of treatment. These compositions can be prepared in a manner well known in the pharmaceutical art, and can be administered by a variety of routes, depending upon whether local or systemic treatment is desired and upon the area to be treated. Administration may be topical (including ophthalmic and to mucous membranes including intranasal, vaginal and rectal delivery), pulmonary (e.g., by inhalation or insufflation of powders or aerosols, including by nebulizer; intratracheal, intranasal, epidermal and transdermal), ocular, oral or parenteral. Methods for ocular delivery can include topical administration (eye drops), subconjunctival, periocular or intravitreal injection or introduction by balloon catheter or ophthalmic inserts surgically placed in the conjunctival sac. Parenteral administration includes intravenous, intra-arterial, subcutaneous, intraperitoneal or intramuscular injection or infusion; or intracranial, e.g., intrathecal or intraventricular administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids, powders, and the like. Conventional pharmaceutical carriers, aqueous, powder or oily bases, thickeners and the like may be necessary or desirable.

### EXAMPLES

### Example 1: Materials and Methods

Human Fetus arrival: Fetal eyes arrive within 24h in 2 degree box. Dissection started directly in order to keep high viability.

Dissection and dissociation: Dissection of the retina was performed on all eyes. Retinas were then dissociated in papain for 30 min in the incubator. Cell suspension was then centrifuged and seeded in a T25 or 175 flask (depending on the number of cells).

Culture for 1-2 weeks: Cells were cultured in a 2D layer for 1-2 weeks using FDA approved media (no animal products). Upon reaching confluence (about 10 million cells) cells were then passaged and centrifuged to start sorting.

First sort with CD73 double positive (PE and APC): Cells were stained with CD73 (PE and APC) for 30 min in 2 degrees. After washing and preparation of the cell sorter, cells are sorted by gating about 3-20% of the positive population. Presort and Postsort analysis was performed.

Culture back for 2 weeks: Positive sorted cells were then cultured in a 175 or T175 for 1-2 weeks until reaching confluence (10 million cells.)

Second sort with CD73/Thrb positive and CDllb negative: The second sort consisted of two different positive antibodies (CD73 and Thrb) and one negative to delete the microglia (CD 11 b). The positive fraction is the new cell line.

Culture to reach high yield: The final cell line was cultured with the same media and flasks to reach the number of cells needed for experiments.

### Results

The first sorting or enrichment step (CD73/CD11b⁻) yielded about 30-40% precursor photoreceptor cells. The second sorting (CD73⁺/THR-beta+/CD11b⁻) for purification of precursor photoreceptor cells yielded a viable 98% pure population of photoreceptor cells with a Ki-67 index of about 20% (proliferation). Accordingly, the method herein has proved to be successful in isolating retinal ganglion cells and retinal microglia, two very difficult targets in the eye.

These cells can be used for cell replacement, drug discovery and screening or other uses where photoreceptors or their precursors might be needed.

This approach has broad applicability for use to isolate other cells in the eye, central nervous system (CNS) and throughout the body.

### Example 2: Cell Culture, Sorting and In vivo Transplantation

Cell Culture

Media 1: Ultraculture media (Lonza)-500 ml; 100x 1-glutamax (Gibco)-5 ml; rhEGF (Peprotech)- 1000 μl(10 μg/l ml); rhFGF (peprotech)- 5000 (1014/m1); Primocin (Invivogen)-1000 μl; Filter and store at 4° C.

Media 2: DMEM/F12- 500 ml; 100x 1-Glutamax 5.5.m1;100x non-essential amino acid- 5.5 ml; Sodium Pyruvate- 5 ml; β-mercaptoethanol- 40; bFGF- 1000u1 Anti-Anti- 5 ml Knock serum replacement (Gibco)- 65m.

For hCPs mix media 1 and 2 in equal quantity just before feeding cells.

Step 1. Prepare papain solution (10 ml per 5 plates)
1. Preparation of fresh activation buffer (1.1 mM EDTA, 0.3 mM β-mercaptoethanol (βME) and 5.5 mM cysteine-HCl).
   50 ml HBSS without Ca, Mg and add the following:
   110 μl of 0.5M EDTA
   0.043 g of L-cysteine Hydrochloride
   1 μl of 14.2βME
2. Papain powder was dissolved to make 0.1 mg/ml of papain: for every 10 ml use 1 mg of papain powder in 10 ml activation buffer; filtered with 0.22 μM filter (syringe/tube);
3. The tube was left open at 37° C., 5% CO2 incubator for 30 min.

Step 2. Eyecup collection (perform during papain activation)
1. Eyecups were collected in the medium in 50 ml tube (from 5-20 plates);
2. Settled by gravity for 2 minutes;
3. The medium was carefully aspirated so as not to lose any eyecups, leave 1 ml;
4. Add 25 ml of HBSS containing 500 μl of anti-anti solution.
5. Carefully tease out retina by removing lens and vitreous fluid and without trace of RPE or ciliary body.
6. Retinas were suspended in 10 ml papain solution for 30 mins at 37° C. to dissociate tissue into single cell suspension.
7. 40 ml of HBSS was added to the tube and centrifuged at 2000 rpm for 5 min.
8. The buffer was aspirated and the pellet was re-suspended in lml media.
9. 10 μl of trypan blue was added to an in Eppendorf tube along with l0 μl of cell suspension.
10. Viability of cells were assessed using automatic cell counter
11. Cell were cultured in fibronectin coated T-75 flask in hypoxia condition till confluency.
   Preparing cells for sorting
12. Cells were lifted from confluent flask using trypsin (1:6 in HBSS).
13. Followed by incubation with trypsin at 37° C. for 3 to 4 min.
14. Cells were collected in 50 ml tube and around 40 ml media containing KSR is added to the cell suspension.
15. Spin at 1200 rpm for 5 min at 15° C.
16. Supernatant was aspirated and cell pellet is suspended in 200 μl of Miltenyi running buffer

### Staining for First Sort

17. Cells were incubated in CD73 APC (Cat no #130-095-183, Miltenyi), CD 73-PE-Vio770 human (Cat no #130-104-192) for 30 minutes on ice.
18. Re-suspend in 10 ml of HBSS and filter using 30 μm filter (Miltenyi).
19. Centrifuge at 300g for 5 mins to wash antibody off.
20. Meanwhile the cell sorting cartridge, was prepped by injecting filtered lml buffer in the input chamber. Pressurized for buffer to move into positive chamber and negative chamber.
21. Buffer was removed from the input chamber concludes cartridge prep.
22. The supernatant was discarded and depending upon cell number, cells were re suspended in 5 to 10 ml (4 to 6 million cells in 5 ml and above 5 million in 10 ml) of MACSQuant Tyto running buffer (Miltenyi) and injected into the input chamber of the cartridge for sorting.
23. 100 μl of the cell suspension was taken in Eppendorf for cell sorting analysis. An example of a cell sorter is the MACSQuant® TYTO® next generation, benchtop cell sorter equipped with 3 lasers, which allows for high speed, 10-parameter cell sorting. A unique feature of the instrument is the fact that the actual sorting process takes place exclusively within a single-use, disposable, and fully closed system. Cell sorting occurs entirely in a closed cartridge that is divided in to three chambers: the input fraction, the sorted fraction and the negative fraction. In a sterile tissue culture flow hood, the user loads the sample in to the sample chamber. Once the cartridge is sealed, nothing goes in or out of it until the sort is complete. The user can then bring the cartridge to the instrument and install it in the sample block. To sort the cells, low air pressure (<3 psi) is applied to the sample, which is then pushed through a fluidic chip. The cells are interrogated by the lasers, the signals are processed, and cells within the population of interest are diverted from the sample stream by a high frequency valve that re-directs the target cell towards the positive chamber. The unsorted flow through continues towards the negative chamber.

### Culturing Post-Sorting

1. Using a fine tip pipet, cells in positive chamber were removed and the chamber was flushed using HBSS.
2. 500 of the cell suspension was placed in an Eppendorf for analysis post-sorting.
3. The rest of the cell suspension is washed with 15 ml of HBSS by centrifuging at 300x for 5 minutes.
4. Cell pellet was resuspended in media (equal quantity of both media) and plated on T 25 flask coated with fibronectin.
5. Incubated at 37° C. at hypoxia condition.
6. Flask was maintained without media change for 48 hrs
7. Followed by depletion of 50% of media, 50% fresh media was added (Media 1 and Media 2 in equal quantity)
8. 2 days post first media change, complete media change was performed.
9. Thereafter every 2 days fresh media was added till confluency was achieved.
10. Confluent flask (should be confluent in 7 to 10 days) was trypsinized and cells were seeded on one t-75 fibronectin coated flask.
11. Media was changed every day till confluency was reached in 3 to 4 days.
12. Cells were trypsinized and spilt in 1:5 flask consistency to continue culture. Or can be used for sorting.

### Preparing Cells for Second Sort:

1. Trypsinized cells were treated exactly same way as first sort.
2. The antibody used in second sorts were: Thyroid Hormone Receptor beta Antibody, ALEXA FLUOR® 647 Conjugated (Cat no # BS-11440R-A647 by BIOSS INC) and CD1lb Vioblue (Cat #130-110-558, Miltenyi) CD 73-PE-Vio770 human (Cat no #130-104-192) for 30 min in ice.
3. Follow same protocol as staining first sort

### Culturing Cells Post-Second Sort:

1. Cells were collected from the output or positive chamber gently using fine pipette tips.
2. Fibronectin coated T-25 flask was used for culturing the sorted cells.
3. Same protocol was followed as first sort.
4. No media change for first 48 hrs. Post this time 50% media depletion and 50% fresh media was added.
5. Cells were cultured in hypoxia condition.
6. Upon confluency flask was trypsinized and cells were seeded on t-75 fibronectin coated flask.
7. Media change every 2 days.
8. One T-75 flask was split in 5-T-75 or 3- T-175 flask.
9. About 40 million cells count is noted after two passage and is frozen until further use.

### NOTES

Retina was dissociated using Papain and should be stiffed once every 15 minutes to ensure complete digestion of the tissue.
T-75 flask was best for culturing freshly dissociated retina. (T-25 would be overcrowding and T-175 would result in sparse cell growth)
Cells were always spilt using trypsin enzyme.
HBSS throughout without calcium and magnesium should be used.
Cells were always maintained in hypoxia.
One T-25 to one T-75 is best after first and second sort. This allows cells to be densely packed without overcrowding.
One T-75 can be spilt 1:5 ratio.
Best to passage cells twice after first sort to obtain 7 to 10 million cells count for the second sort.
7 passage is maximum these cells have been expanded after second sorted cells.

TABLE 1
Sorting Data

| | ID | Weeks | Date | Cells | Singles | PreSort CD73 | PostSort CD73 | Neg CD73 |
|---|---|---|---|---|---|---|---|---|
| 1st | 642301 | 14 | Aug. 24, 2018 | 94.82 | 93.1 | 3.39 | 79.82 | 1.31 |
| SORTS | 731101 | 12 | Sep. 12, 2018 | 99.45 | 98.17 | 3.12 | 64.54 | 2.1 |
| | 734901 | 13 | Oct. 19, 2018 | 78.92 | 62.28 | 26.37 | 77.3 | 21.57 |
| | 310403 | 16 | Oct. 19, 2018 | 90.23 | 75.71 | 30.07 | 80.98 | 26.23 |
| | 311701 | 16 | Jan. 28, 2019 | 90.23 | 86 | 34.12 | 82.78 | 14.12 |
| | 731102 | 16 | Apr. 19, 2019 | 96 | 82 | 70.16 | 96.98 | 37.25 |
| | 311802 | 16 | Apr. 25, 2019 | 95 | 87 | 70.68 | 84.25 | 57.45 |
| | 311802 | 16 | Apr. 25, 2019 | 95 | 87 | 72.81 | 95.63 | 40.15 |
| | 730701 | 14 | May 13, 2019 | 94.82 | 79 | 48.27 | 87.1 | 38.15 |
| | 310901 | 12 | May 15, 2019 | 94 | 78 | 29.84 | 89.25 | 21.98 |

| | ID | Weeks | Date | Cells | Singles | PreSort CD73/Thrb | PostSort CD73/Thrb | Neg CD73/Thrb |
|---|---|---|---|---|---|---|---|---|
| 2nd | 642301 | 14 | Oct. 22, 2018 | 72.82 | 64.67 | 56.71 | 83.63 | 27.25 |
| SORTS | 642301 | 14 | Oct. 22, 2018 | 66.91 | 62.21 | 40.05 | 88.25 | 28.2 |
| | 731101 | 12 | Oct. 22, 2018 | 85 | 72 | 61.45 | 75.38 | 41.25 |
| | 734901 | 13 | Nov. 12, 2018 | 84.43 | 76.63 | 74.62 | 92.49 | 25.3 |
| | 311701 | 16 | Feb. 8, 2019 | 84.46 | 73.78 | 54.78 | 93.92 | 22.1 |
| | 731102 | 16 | Apr. 23, 2019 | 88 | 98 | 20.45 | 91.87 | 8.12 |
| | 311802 | 14 | May 13, 2019 | 90.23 | 79 | 47.85 | 97.15 | 12.65 |
| | 730701 | 14 | May 27, 2019 | 94.82 | 85 | 45.03 | 94.85 | 12.43 |
| | 310901 | 12 | May 27, 2019 | 94 | 82 | 38.45 | 93.65 | 17.85 |

TABLE 2
Flow Cytometry Data

| | | | | | |
|---|---|---|---|---|---|
| [symbol] | [symbol] | [symbol] | [symbol] | [symbol] | [symbol] |

[symbol]

Single sort (2 weeks [symbol] )

| | | | | | |
|---|---|---|---|---|---|
| [symbol] | [symbol] | 5 [symbol] | 57 [symbol] | | 0.02 |
| [symbol] | [symbol] | [symbol] | [symbol] .23 | | [symbol] .7 |
| [symbol] | [symbol] | 65. [symbol] 2 | [symbol] .55 | | [symbol] |
| [symbol] | 86. [symbol] | [symbol] | [symbol] 7.22 | | [symbol] |
| [symbol] | [symbol] | [symbol] | [symbol] | | [symbol] |
| [symbol] | 88.72 | [symbol] 4 | [symbol] | | [symbol] |
| [symbol] | [symbol] | [symbol] | [symbol] | 2.00 | |
| [symbol] 67 | [symbol] 6.77 | [symbol] | 55. [symbol] | [symbol] | |
| [symbol] | [symbol] | 6 [symbol] 2 | 57.5 [symbol] | [symbol] | |

[symbol]

Db sort Positive (2 weeks [symbol] )

| | | | | | |
|---|---|---|---|---|---|
| [symbol] | [symbol] .14 | 75.96 | 72.72 | | [symbol] 05 |
| [symbol] | [symbol] 4.55 | 78.46 | 75.22 | | 2. [symbol] |
| [symbol] | [symbol] 1 | [symbol] 52 | 77.88 | | [symbol] |
| [symbol] | [symbol] 5 [symbol] | [symbol] 78. [symbol] | 7 [symbol] 3 | | [symbol] |
| [symbol] | [symbol] .2 | [symbol] 4 | 7 [symbol] | | [symbol] |
| [symbol] | [symbol] .5 | [symbol] | [symbol] | | [symbol] .00 |
| [symbol] | [symbol] | [symbol] .47 | [symbol] .88 | [symbol] | |
| [symbol] | [symbol] .2 | 73. [symbol] | [symbol] 8.02 | 5 [symbol] .77 | |
| [symbol] | [symbol] .7 | 78.4 [symbol] | 7 [symbol] 7 | 6.54 | |

[symbol]

sort Negative (2 weeks [symbol] )

| | | | | | |
|---|---|---|---|---|---|
| [symbol] | [symbol] | [symbol] | [symbol] | | [symbol] |
| [symbol] | [symbol] | [symbol] | [symbol] 55 | | [symbol] .27 |
| [symbol] | [symbol] | [symbol] | [symbol] | | [symbol] |
| [symbol] | [symbol] 66 | [symbol] | [symbol] 2.59 | | [symbol] .41 |
| [symbol] | [symbol] 7 [symbol] | [symbol] | [symbol] .2 [symbol] | | 22. [symbol] 5 |
| [symbol] | [symbol] | [symbol] | [symbol] 2.4 [symbol] | | [symbol] |
| [symbol] | [symbol] | [symbol] | [symbol] | 2. [symbol] | |
| [symbol] | [symbol] | [symbol] 7 [symbol] | [symbol] | [symbol] | |
| [symbol] | [symbol] | 87 [symbol] 5 | [symbol] | [symbol] .27 | |

[symbol]

sort Positive (2 weeks [symbol] )

| | | | | | |
|---|---|---|---|---|---|
| [symbol] | [symbol] | [symbol] | [symbol] .25 | | [symbol] |
| [symbol] | [symbol] | 5 [symbol] 48 | [symbol] | | 2.05 |
| [symbol] | [symbol] .42 | 74. [symbol] | 7 [symbol] 7 | | 84. [symbol] |
| [symbol] | [symbol] .28 | [symbol] | [symbol] | | [symbol] .52 |
| [symbol] | [symbol] .23 | [symbol] .79 | [symbol] 7.22 | | [symbol] .41 |
| [symbol] | 87. [symbol] | [symbol] | [symbol] | | [symbol] |
| [symbol] | 88.2 [symbol] | [symbol] 7 | [symbol] .02 | [symbol] 04 | |
| [symbol] | [symbol] 4. [symbol] | [symbol] | [symbol] | 32. [symbol] | |
| [symbol] | 85. [symbol] | [symbol] .8 [symbol] | [symbol] .0 [symbol] | [symbol] .02 | |

[symbol]

Positive ( [symbol] weeks [symbol] )

| | | | | | |
|---|---|---|---|---|---|
| [symbol] | [symbol] | 5 [symbol] .26 | [symbol] 7.7 | | [symbol] |
| [symbol] | 55.72 | [symbol] 2.74 | [symbol] .35 | | 5.52 |
| [symbol] | [symbol] | 75. [symbol] | 7 [symbol] | | 0.2 |
| [symbol] | 5 [symbol] | 72. [symbol] | [symbol] | | [symbol] .19 |
| [symbol] | 22. [symbol] | 55. [symbol] | [symbol] | | 9 [symbol] |
| [symbol] | [symbol] | [symbol] | [symbol] | | [symbol] .19 |
| [symbol] | [symbol] | [symbol] | 75.4 [symbol] | | 0. [symbol] |
| [symbol] | [symbol] | [symbol] .27 | [symbol] | | [symbol] .0 [symbol] |
| [symbol] | [symbol] | [symbol] | [symbol] | | 20. [symbol] |
| [symbol] | [symbol] | 75. [symbol] | [symbol] 2 | | [symbol] .27 |
| [symbol] | [symbol] 4 | [symbol] | [symbol] .20 | 2.0 [symbol] | |
| [symbol] | [symbol] | [symbol] | 55.8 [symbol] | [symbol] .87 | |

[symbol]

Db Unsorted

| | | | | | |
|---|---|---|---|---|---|
| [symbol] | [symbol] .2 | [symbol] | [symbol] | | [symbol] |
| [symbol] | [symbol] | [symbol] | [symbol] | | 2. [symbol] |
| [symbol] | [symbol] | 7 [symbol] 2 | [symbol] | | |
| [symbol] | [symbol] .01 | [symbol] .5 | 75. [symbol] | | [symbol] |
| [symbol] | [symbol] | [symbol] | [symbol] | | [symbol] |
| [symbol] | [symbol] 2 | [symbol] | 8 [symbol] | | [symbol] |
| [symbol] | [symbol] | [symbol] .27 | [symbol] 2. [symbol] | | [symbol] |
| [symbol] | [symbol] .73 | 63. [symbol] | [symbol] | | [symbol] .06 |
| [symbol] | [symbol] | [symbol] | [symbol] | | [symbol] |
| [symbol] | 90 [symbol] 5 | 6 [symbol] | [symbol] .55 | | 0.2 |
| [symbol] | [symbol] 6 [symbol] | [symbol] | 27. [symbol] | 2. [symbol] | |
| [symbol] | [symbol] 8. [symbol] 2 | [symbol] | [symbol] | [symbol] .21 | |

[symbol]

Db Unsorted

[symbol]

| | | | | | |
|---|---|---|---|---|---|
| [symbol] | [symbol] | [symbol] | [symbol] | | 0.02 |
| [symbol] | [symbol] .72 | [symbol] .74 | [symbol] .35 | | 2.17 |
| [symbol] | [symbol] | [symbol] .81 | [symbol] | | |
| [symbol] | [symbol] | [symbol] | [symbol] .83 | | [symbol] .28 |
| [symbol] | [symbol] | [symbol] | [symbol] .01 | | [symbol] 1. [symbol] |
| [symbol] | [symbol] | 78 [symbol] | [symbol] | | [symbol] .06 |
| [symbol] | [symbol] | [symbol] 0.8 [symbol] | [symbol] .25 | | 5. [symbol] |
| [symbol] | [symbol] | [symbol] 8 [symbol] | [symbol] 88 | | 8. [symbol] |
| [symbol] | [symbol] | 8 [symbol] | [symbol] .40 | | 4 [symbol] |
| [symbol] | [symbol] .2 | [symbol] 8 | [symbol] 2 | | 0.3 [symbol] |
| [symbol] | [symbol] 6.1 | 7 [symbol] 7 | 7 [symbol] | [symbol] .23 | |
| [symbol] | [symbol] | 73.9 [symbol] | [symbol] .32 | [symbol] 9 | |

[symbol]

Db sort Positive

| | | | | | |
|---|---|---|---|---|---|
| [symbol] | [symbol] | [symbol] 27 | [symbol] | | 0.0 [symbol] |
| [symbol] | [symbol] | [symbol] | [symbol] | | 2.17 |
| [symbol] | [symbol] .2 | [symbol] | [symbol] .02 | | [symbol] |
| [symbol] | [symbol] | [symbol] | [symbol] | | [symbol] .2 |
| [symbol] | [symbol] .1 | [symbol] | [symbol] .23 | | [symbol] |
| [symbol] | [symbol] | 7 [symbol] | [symbol] | | [symbol] |
| [symbol] | 55 [symbol] | 22. [symbol] | [symbol] .35 | | [symbol] |
| [symbol] | 2 [symbol] | [symbol] | 22. [symbol] 9 | | [symbol] |
| [symbol] | 5 [symbol] 2 | [symbol] | [symbol] 7 | | 37. [symbol] 7 |
| [symbol] | 22. [symbol] | 55.7 [symbol] | [symbol] | | 0.86 |
| [symbol] | [symbol] .04 | [symbol] .82 | [symbol] | 2.2 [symbol] | |
| [symbol] | [symbol] | [symbol] | [symbol] .83 | [symbol] 4.8 [symbol] | |

[symbol]

indicates data missing or illegible when filed

TABLE 3A
Transplantation study
CD73/Thrb 15 w 4/23

| Rat | | Ear | | Injection | |
|---|---|---|---|---|---|
| Number | Eye | punch | Cage | space | Comments |
| #1 | Right | Right | e | subretinal | good injection |
| #2 | Right | Right | e | subretinal | good injection |
| #3 | Right | Right | e | | Retinal damage |
| #4 | Right | Right | f | | Retinal damage |
| #1 | Right | Right | f | | good injection |
| #2 | Right | Right | f | subretinal | good injection |
| #3 | Right | Right | g | subretinal | good injection |
| #4 | Right | Right | g | subretinal | good injection |
| #1 | Right | Right | g | subretinal | good injection |

| Viability | Time | 15 w | 16 w |
|---|---|---|---|
| | 0 | 95% | 93% |
| | 0.5 | 93% | 92% |
| | 1 | 91.5% | 91% |
| | 2 | 89% | 90% |
| | 3 | 83% | 85% |
| | 4 | 82% | 83% |

TABLE 3B
CD73/Thrb 16 w 4/12

| Rat | | Ear | | Injection | |
|---|---|---|---|---|---|
| Number | Eye | punch | Cage | space | Comments |
| #1 | Right | Right | a | subretinal | good injection |
| #2 | Right | Right | a | subretinal | good injection |
| #3 | Right | Right | a | subretinal | good injection |
| #4 | Right | Right | b | subretinal | good injection |
| #1 | Right | Right | b | | Damage |
| #2 | Right | Right | b | subretinal | good injection |
| #3 | Right | Right | c | subretinal | good injection |
| #4 | Right | Right | c | subretinal | good injection |
| #1 | Right | Right | c | | Damage |
| #2 | Right | Right | d | subretinal | good injection |
| #3 | Right | Right | d | | No cells |
| #4 | Right | Right | d | subretinal | good injection |

| | | | |
|---|---|---|---|
| Injection 16 w | Jun. 25, 2019 | Injection 15 w | Jun. 28, 2019 |
| End of exp | Jul. 9, 2019 | End of exp | Jul. 11, 2019 |

TABLE 3C

| Weeks of gestation | # Transplanted | Cell Survival | Cell Engraftment |
|---|---|---|---|
| 15 weeks | n = 11 | n = 11 (100%) | n = 10 (91%) |
| 16 weeks | n = 9 | n = 9 (100%) | n = 8 (88%) |

Sort

Cells were analyzed with a flow cytometer prior to sorting which are set up as the control for running the sort. Markers of choice (depending on the sort), CD73, Thrb and CD11b are analyzed with the flow cytometer by tuning the voltages in order to put the desired population in the highest quadrant of the intensity. After this first gating strategy was performed, the the microfluidic flow is started.

Cells were first pushed through the negative chambers in order to stabilize the pressure (around 150 mPa) but also the flow of cells in front of the lasers (40 ms between two cells). In order for the machine to know its velocity, a cell must be stained in at least two different fluorochromes, this way the machine can calculate the time it takes the cell to go from the first laser to the second one, hence measuring its velocity. As soon as both these variables are stable, lasers can be started and fluorescent data starts to appear showing an approximate of 5000-7000 cells that are being pushed in front of the lasers. Each cell passes in front of the three lasers and its fluorescent intensity is measured and reported on the graph.

The same gating strategy that was used on the control on the cell sorter and reported on the sorting machine and the sort was started. However, in order to capture properly each cell, the valve speed and delay of action must be properly set up. The arrival window was open and was then fitted to the exact population of cells flowing through the microfluidic device (this depends on cell size, shape, granularity, intensity, stiffness, concentration).

During the entire sorting process (usually 2 hours) the percent of sorted, gated and positive cells was measured and reported in function of time. At the end of the sort, the cartridge was freed from the machine and cells can be taken back to the cell culture for next steps.

Flow Cytometry

hCPs [(5×10⁵/mL in media) were trypsinized and cell pellet collected was processed for the phenotype then analyzed using a Flow Cytometry assay.

Flow Cytometry was performed using MACSQuant flow cytometer (Miltenyi, San Diego) (100). Cells were collected and fixed with Perm/Fix buffer (BD Biosciences) at 4° C. for 15 min. Cells were then washed in wash buffer (BD Biosciences) and incubated, at room temperature, in block buffer (Pharmingen staining buffer with 2% goat serum) for 30 min. Blocked cells were seeded onto a flat bottom 96-well plate (treated, sterile, polystyrene, Thomas Scientific) and stained with conjugated primary antibodies (DAPI-Vioblue, Cone Arrestin-FITC, S-opsin-FITC, R/G opsin-FITC, Blue opsin- Rhodopsin-FITC, Recoverin-APC, Calbidin- FITC, RBPMS-APC, PKCa-FITC and Brna3a-FITC, KI67-APC) overnight at room temperature. Primary antibodies were diluted in 200 μL of antibody buffer (TBS, 0.3% Triton X-100 and 1% goat serum). After cells were washed three times for 15 min, secondary antibodies were goat-derived anti-rabbit and anti-mouse and diluted 1:200 in antibody buffer (Jackson Immunoresearch Laboratory). Secondary antibodies were applied and left at room temperature for 3 h. light scatter and fluorescence signals from each well were measured using the MACSQuant flow cytometer (2×10⁵ events were recorded). The results were analyzed using the MACSQuantify software (Miltenyi Biotec). For each primary antibody DAPI-positive single cell population was gated. The ratio of positive cells in the gated population was estimated in comparison with blank and species-specific isotype control.

Immunohistochemistry (IHC)

Live hCPs grown in chamber slides and cryosection from Long Evans left eye were fixed with 4% paraformaldehyde in 0.1 M PBS (Irvine Scientific) at room temperature for 20 min. These fixed cells and sections were blocked and permeabilized with a blocking solution [(iris-buffered saline (TBS), 0.3% Triton X-100 and 3% goat serum (Jackson Immunoresearch Laboratories, West Grove, Pa.) for 15 min. Samples were then rinsed twice with 0.1 M TBS buffer for 15 min each time, mounted on polysine microscope slides and incubated with primary antibodies overnight at 4° C. (DAPI-Vioblue, Cone Anrestin-CY3, R/G opsin-Cy3, TRA-1-85- FITC, STEM 121-FITC) at concentrations determined in laboratory. The next day, samples were rinsed three times with TBS for 15 min. Secondary antibodies (goat-derived anti-mouse and anti-rabbit) were applied for lh at room temperature. Samples were then washed one last time with TBS before being mounted on polysine microscope slide with lox viscosity slide mounting medium. Digital images were obtained with an Epifluroscent microscope using 20x objective. Electronic image files were managed using Matlab software.

Quantitative Real-Time RT-PCR (aRT²-PCR)

RNA extraction was performed using the Qiagen kit. Cells were treated with trypsin and pellet collected was disrupted using Buffer RLT (1 ml of RLT buffer add l00 of β-mercaptoethanol) and the tube was flicked to disrupt the cells. One volume of 70% ethanol (not provided in the kit) was added and mixed well by pipetting. The suspension was transferred into the RNeasy spin column (provided in the kit). Close lid and gently centrifuged at 8000g for 15 seconds. The flow through was discarded and about 7000 of RW1 buffer was added and then spun for 15 sec at 8000 x g. The flow through was discarded and 5000 of RPE buffer (both buffers provided in the kit; RPE buffer add ethanol before using) was added and then spun for 15 sec at 8000 x g. This was followed by second wash with RPE buffer for 2 min at 8000 x g. The flow through was discarded and the spin column was placed in a new 1.5 ml collection tube and RNA was eluted by gently adding RNAase free water directly to the spin column. The lid was closed and centrifuged for 1 in at 8000 x g. RNA was quantified using nanodrop and 1 μg of total RNA was used for cDNA synthesis using iScript cDNA advance transcription kit (Bio-rad).

| Reagent | Volume | |
|---|---|---|
| iScript Reverse transcriptase | 4 | μl |
| 5x reaction buffer | 1 | μl |
| cDNA | 1 or 2 | μg |

| | |
|---|---|
| Water | variable |

Total reaction volume=200

Reaction protocol used: Priming 5 min at 25° C. followed by reverse transcription step for 20 mins at 46° C. and finally RT inactivation for 1 min at 95° C. Synthesized cDNA was used for gene expression analysis.

| Reagent | Volume | |
|---|---|---|
| 2x SYBR Green | 10 | μl |
| cDNA | 10-20 | ng |

| | |
|---|---|
| Water | variable |

The volume was calculated for 96 well plates and data was quantified using Biorad software.

In-Vivo Transplantation

Twenty-three male domestic rats of the Long Evans breed (age 12 weeks, approximate weight 200g) were used as recipients in the experiment. Prior to surgery, all animals were pre-anesthetized with GAS. Rats were anesthetized with 2%-3% isoflurane (Abbott, Solna, Sweden) in combination with oxygen. Transplantation was performed on non-immuno-suppressant rats. Rats were sedated using 2-4% isoflurane by placing the rats in the inhalation chamber, followed by intraperitoneal injection of ketamine (40-80 mg/kg) and xyalzine (10 mg/kg) for anesthesia. Eyes were first anesthetized using topical ophthalmic proparacacine (0.5%) followed by Genteal to keep the lens moist during the surgery.

Recipient rats were injected in sub-retinal space with hCP single-cells injections. A conjunctival incision and a small sclerotomy were performed using a fine disposal scalpel. Cells were injected into the subretinal space using a glass pipette (internal diameter, 150 urn) attached to a 50-μL Hamilton syringe via a polyethylene tubing. The hCPs were injected into the retina bleb as a single-cell suspension in PBS. All samples contained approximately 1×10⁵ cells and the injection volume were 2 μL for all replicates. Using a glass coverslip applied on the eye checked bleb presence. Subretinal space injection was considered successful is a shining bleb was seen under the dissection surgical microscope. Triple antibiotic (Bac/Neo/Poly) was given locally at the end of the surgery to prevent further infection. The rats were then placed in their cages for a 14 days study.

The research protocol was reviewed and approved by the Schepens Eye Research Institute Animal Facility and was in accordance with the Association for Research in Vision Ophthalmology Statement for the Use of Animals in Ophthalmic and Vision Research.

Tissue Processing

Three Days post transplantation rats were sacrificed by CO2 suffocation for 2 min. Eyes were enucleated and placed in 4% paraformaldehyde for 24 hrs. Tissues were subsequently saturated with increase concentration of sucrose (5%, 10%, 20%) containing Sorensen phosphate buffer. Eyes were left in 30% sucrose overnight or till dissection. The tissues were embedded in cryo-section gelatin medium overnight and sectioned at 15 μm thickness on a cryostat. During the sectioning process, every 5th section was stained and examined by epi-fluorescence for hCPs presence with TRA-1-85 and STEM 121-FITC (human cells marker), R/G opsin and Cone Anrestin-APC (host photoreceptor marker) and DAPI-Vioblue (cell nuclei).

Micro-Electrode Array Assay

Using a 1 mL pipette tip, a 2 mL cell suspension containing around 500,000 hCP was placed on the MaxWell MaxOne Multielectrode Array and aligned to the electrode. 16,000 electrodes on 4 mm² was used. A 5-mm coverslip was placed between the microscope objective and the cell suspension to maintain an optically aberration-free transition zone from the air to the liquid. The HD-MEA chip was plugged into the interfacing circuit board. The cells were allowed to acclimate to the MEA under a 50% contrast background for 20 min.

The data recorded on the MEA was sampled at 20 kHz, and filtered on-chip, approximately between 0.5 Hz and 12 kHz. Data was then filtered with a 280 Hz high-pass filter and 7 kHz low-pass filter to reduce offset effect and high frequency noises.

Light stimuli were programmed and sent to a LED projector. The projected image was centered on the selected electrode region, and light stimuli were run sequentially. Prior to each stimulus, a 50% contrast background was projected onto the cell suspension for 5 min so that the cells could adapt to the mean projected photopic level.

### Example 3: Clinical Trial Procedure

Frozen formulation delivered to clinical site (alternatively cells delivered in culture vessels at 37 degrees C.).

Direct injection of 50,000-10×10⁶ cells into subretinal under macula.

3 port pars plana vitrectomy approach, or delivered with device aimed at subretinal space.

No systemic immunosupression (local anti-inflammatory treatment) anticipated. Cone dystrophy, an orphan indication with no available treatments:

Phase 1/2a in patients <20/200 without significant cones in macula but with preserved outer retinal structures (RPEBM/CC) first for safety.

Progressing through subsequent phases into patients with better function.

Outcomes and assessment: Visual acuity (VA) for functional and ophthalmic coherence tomography (OCT) for structural evaluation.

Other more novel outcome measures (e.g. ellipsoid zone (EZ) on OCT) expected to be available by proposed trial date.

Followed by dry AMD, other indication where cone structure and function is impaired (e.g. Retinal Detachment).

### Example 4: Isolation, Enrichment and Transplantation of Rare Human Cone Progenitor Cells for Treatment of Cone Dystrophy

Stargardt's and Cone dystrophies are retinal diseases caused by a gene mutation in cone photoreceptors, which leads to death of these cells. In early stages of these diseases, gene therapy is useful for treatment; however, no treatment is available for later stages of these diseases. The methods and cells described herein provide a solution to a longstanding clinical problem in treating theses pathologies. Rare cone progenitors cells (hCP) were isolated and enriched from human fetal embryonic retinal tissue. For stem cell-based therapy it is crucial to obtain a purified highly enriched, e.g., at least 85% pure (e.g., 90, 91, 92, 93, 94, 95, 96, 97, 98, 99, 100% pure) or a homogenous cell population. For example, 95% pure cone progenitor cells were achieved using a 2 step sorting technique. Fetal retina was first dissociated and cultured to obtain the desired cell number of 10M. The first sorting step involved tagging the cells with CD73 surface antigen, which is exclusively expressed in the photoreceptor precursor population. The positive cells were further cultured in hypoxia conditions (10% oxygen) and sorted with CD73⁺Thrb⁺ and CD 11 b- cells using Miltenyi Tyto cell sorter. Double positive staining of cells ensured higher purity with CD11b- acting as negative channel that allowed us to delete specific cell population (i.e. microglia). These cells could be cultured and expanded in hypoxia conditions. The phenotypical expression of these cells was confirmed using a MacsQuant cell analyzer and identified these cells as human cone progenitor cells after testing for cone arrestin, s-opsin, m-opsin and blue-opsin expression (95%). These cells were found to have low PKCa expression (4%), and no rhodopsin, Brn3a, NeuN, and RBPMS indicating the purity of these cells with few contaminating cells of non-cone lineage. This strategy to isolate, enrich and culture was used to obtain large number of hCPs which were transplanted into rat eyes showing extremely high capacity to survive and engraftment into the host inner retina. The cells are also evaluated in photoreceptor degeneration rat models. The data demonstrated the ability of hCP to integrate, survive and engraft in a xenograft model indicating that the purified cell population is useful to treat retinal diseases such as cone dystrophies and/or photoreceptor degenerative conditions.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

1. A composition comprising a purified population of cells, wherein the cells are CD73⁺, Thyroid Hormone Receptor beta (Thrb⁺), CD 11 b⁻.

2. The composition of claim 1, wherein the purified population of cells are derived from: embryonic retinas, embryonic retinal tissues, embryonic stem cells, mesenchymal stem cells, induced pluripotent stem cells (iPSCs), or iPSC-derived retinal organoids.

3. The composition of claim 2, wherein the purified population of cells are derived from embryonic retinas.

4. The composition of claim 1, wherein the purified population of cells comprise at least 50% of a total number of cells in the composition having an expression marker profile of CD73⁺, Thrb⁺, CD 11 b⁻.

5. The composition of claim 1, wherein the purified population of cells comprise at least 75% of a total number of cells in the composition having an expression marker profile of CD73⁺, Thrb⁺, CD 11 b⁻.

6. The composition of claim 1, wherein the purified population of cells comprise at least 80% of a total number of cells in the composition having an expression marker profile of CD73⁺, Thrb⁺, CD 11 b⁻.

7. The composition of claim 1, wherein the purified population of cells comprise at least 85% of a total number of cells in the composition having an expression marker profile of CD73⁺, Thrb⁺, CD 11 b⁻.

8. The composition of claim 1, wherein the purified population of cells comprise at least 90% of a total number of cells in the composition having an expression marker profile of CD73⁺, Thrb⁺, CD 11 b⁻.

9. The composition of claim 1, wherein the purified population of cells comprise at least 95% of a total number of cells in the composition having an expression marker profile of CD73⁺, Thrb⁺, CD 11 b⁻.

10. A method of producing progenitor photoreceptor cells, the method comprising:
culturing retinal progenitor cells;
isolating CD73⁺cells from the cultured retinal progenitor cells;
culturing CD73⁺cells;
subjecting the CD73⁺cells to a second isolation step comprising isolating CD73⁺Thrb⁺ and CD11b⁻cells;
culturing and expanding the CD73⁺Thrb⁺CD 11 b⁻cells; thereby,
producing the progenitor photoreceptor cells.

11. The method of claim 10, wherein the retinal progenitor cells are derived from embryonic retinas, embryonic stem cells, mesenchymal stem cells, induced pluripotent stem cells (iPSCs), or iPSC-derived retinal organoids.

12. The method of claim 11, wherein the retinal progenitor cells are derived from embryonic retinas or embryonic retinal tissues.

13. The method of claim 12, wherein the embryonic retinas or retinal fetal tissues are contacted with an enzyme to obtain a cell suspension.

14. The method of claim 10, wherein the CD73⁺Thrb⁺CD11b⁻cells comprise at least 90% of the total cell counts.

15. The method of claim 10, wherein the CD73⁺Thrb⁺CD11b⁻cells comprise at least 95% of the total cell counts.

16. A method of producing progenitor photoreceptor cells, the method comprising:
obtaining embryonic retinas or retinal tissues and dissociating the embryonic retinas or retinal tissue with an enzyme to produce a cell suspension;
culturing cells obtained from the cell suspension;
isolating CD73⁺cells from the cell culture and further culturing CD73⁺cells;
subjecting the CD73⁺cells to a second isolation step comprising isolating CD73⁺Thrb⁺ and CD11b⁻cells;
culturing and expanding the CD73⁺Thrb⁺CD 11 b⁻cells; thereby,
producing the progenitor photoreceptor cells.

17. The method of claim 16, further comprising culturing the progenitor photoreceptor cells with one or more agents or culturing conditions.

18. The method of claim 17, wherein the one or more agents comprise growth factors, cytokines, reprogramming factors, hormones, cells, tissues or combinations thereof.

19. The method of claim 17, wherein the culturing conditions comprise: culturing substrates, co-culturing environment, two- or three-dimensional culturing.

20. The method of claim 16, wherein the progenitor photoreceptor cells differentiate into cone photoreceptor cells.

21. The method of claim 20, wherein the cone photoreceptor cells identified by markers comprising: Cone Arrestin+, Red/G opsin⁺Rhodopsin⁻.

22. A method of producing a purified population of progenitor cells, the method comprising:
obtaining or isolating cells from a biological sample;
culturing and expanding the cells;
isolating cells based on a first biomarker profile and further culturing of the isolated cells;
subjecting the cultured isolated cells to a second isolation step based on a second biomarker profile; thereby,
producing a purified population of progenitor cells.

23. The method of claim 22, wherein the biological sample comprises: fetal tissues, embryonic tissues, extraembryonic, tissues, cord blood, cord tissues, fluids, bone marrow, adult tissues or combinations thereof.

24. A method of treating an ocular or retinal disease comprising administering to a subject an effective amount of the composition of claim 1.

25. A method of screening for a candidate therapeutic agent comprising: contacting a cell of claim 1, with a candidate therapeutic agent;
comparing genotypic and/or phenotypic characteristics and/or induction of differentiation of the cell of claim 1 to a baseline control in the presence or absence of the candidate therapeutic agent and correlate characteristics of a certain disease to specific genetic or phenotypic features.
